(19) United States
(12) Patent Application Publication
(10) Pub. No.: US 2026/0034403 A1
(43) Pub. Date: Feb. 5, 2026

(54) SYSTEM AND METHOD FOR REAL-TIME FITNESS TRACKING AND SCHEDULING

(71) Applicant: Timothy Coyle, New York, NY (US)

(72) Inventor: Timothy Coyle, New York, NY (US)

(21) Appl. No.: 19/358,478

(22) Filed: Oct. 15, 2025

Related U.S. Application Data
(63) Continuation-in-part of application No. 18/884,242, filed on Sep. 13, 2024.
Application No. 18/884,242 is a continuation-in-part of application No. 17/154,349, filed on Jan. 21, 2021.
(60) Provisional application No. 62/964,172, filed on Jan. 22, 2020.

(51) Int. Cl. A63B 24/00; A61B 5/00; A61B 5/0205; A63B 71/06; G06Q 10/1093; G16H 10/20

(52) U.S. Cl. CPC A63B 24/0062; A61B 5/0002; A61B 5/0205; A61B 5/4824; G06Q 10/1093; G16H 10/20; A63B 2024/0068; A63B 2024/0078; A63B 2071/065; A63B 2230/04; A63B 2230/75

(57) ABSTRACT

A computer system configured to implement a method for real-time fitness tracking and scheduling is described herein. The computer system receives health data, a first health goal for completion during a first time period, and a second health goal for completion during a second time period for a user. A health profile is generated that includes the health data, the first health goal, and the second health goal. The health data is implemented in if-then scenarios to determine a first wellness action for the user to complete during a first time period to achieve the first health goal and a second wellness action for the user to complete during a second time period to achieve the second health goal. A health and wellness program is created for the user based on the first wellness action and the second wellness action.

### CROSS-REFERENCE TO RELATED APPLICATIONS

This application is a U.S. Continuation-In-Part Utility Patent Application entitled, “SYSTEM AND METHOD FOR REAL-TIME FITNESS TRACKING AND SCHEDULING” which claims priority to co-pending U.S. Continuation-In-Part Utility patent application Ser. No. 18/884,242 entitled, “SYSTEM AND METHOD FOR REAL-TIME FITNESS TRACKING AND SCHEDULING” filed on Sep. 13, 2024, which claims priority to U.S. Non-Provisional Utility patent application Ser. No. 17/154,349 entitled, “SYSTEM AND METHOD FOR REAL-TIME FITNESS TRACKING AND SCHEDULING” filed on Jan. 21, 2021 which claims priority to U.S. Provisional Patent Application No. 62/964,172, filed on Jan. 22, 2020, the contents of which are hereby fully incorporated by reference.

### FIELD OF THE EMBODIMENTS

The field of the invention and its embodiments relate to a method for real-time fitness tracking and scheduling. In particular, the present invention and its embodiments provide a method for real-time fitness tracking and scheduling that incorporates, via an algorithm, health data of a user in if-then scenarios to determine user-specific wellness actions to complete to achieve health goals and adapt to provide new goals, new suggestions, and new guidance over time. Moreover, the present invention and its embodiments provide a method for real-time fitness tracking and scheduling that converts to unique actionable recommendations that evolve and adapt to the user's input over time.

### BACKGROUND

A person's health is a combination of multiple factors, including medical data (such as a known health problem of the user, a health problem of a family member associated with the user, 2 5 10 a physiological or biochemical measurement of the user, etc.), genetic data (such as genomic information), nutritional data (such as types of foods eaten by the user, a number of daily calories consumed by the user, a quantity of meals consumed daily by the user, etc.), fitness data (such as a type of exercise routine engaged in by the user, a type of workout engaged in by the user, a length of time spent on the exercise routine, a number of calories burned during the workout, etc.), and environmental data (such as lifestyle choices of a user). Health and fitness professionals typically assess only one or two of these factors when creating a health and wellness program for an individual to achieve a health goal. For example, a doctor may only assess medical data associated with a user, whereas a physical trainer may only assess fitness data associated with the user when creating a health and wellness program for the user. Thus, current solutions fail to adequately assess the relationships between these factors and also fail to generate or provide a complete health and wellness program for an individual to achieve one or more health goals. Thus, a need exists for a method for real-time fitness tracking and scheduling that incorporates, via an algorithm, health data of a user in if-then scenarios to determine user15 specific wellness actions to complete to achieve health goals.

Various methods for real-time fitness tracking and scheduling are known in the art. However, their means of operation are substantially different from the present disclosure, as the other inventions fail to solve all the problems taught by the present disclosure. The present invention and its embodiments provide a method for real-time fitness tracking and scheduling. In particular, the present invention and its embodiments provide a method for real-time fitness tracking and scheduling that that incorporates, via an algorithm, health data of a user in if-then scenarios to determine user-specific wellness actions to complete to achieve health goals.

### Summary of the Embodiments

In some aspects, the techniques described herein relate to a method executed by a health engine of a computing device for real-time fitness tracking and scheduling, the method including: providing the computing device including: an output device having a graphics processing unit in bidirectional communication with an interface bus; an interface controller in bidirectional communication with the interface bus; one or more data storage devices in communication with a health engine via the interface controller; and a peripheral interface having a serial interface controller in communication with a parallel interface controller, the parallel interface is in bidirectional communication with the interface bus; receiving, by the health engine, health data being at least a textual description of at least one of a location and an intensity of pain; receiving, from one or more wireless health devices, real-time biometric data of a user including at least one of a heart rate measurement, blood pressure measurement, or calories burned during a workout; displaying, via a graphical user interface (GUI) of the computing device, a graphic of the pain as pain points on a graphical representation of a human body based on an analysis by the health engine of the textual description of the pain; receiving, by the health engine, a first health goal of the user for completion during a first time period and a second health goal of the user for completion during a second time period, wherein the health data, the first health goal, and the second health goal are received from user input in response to a health questionnaire, wherein the first health goal is relieving pain, wherein the second health goal is to complete a sports event, wherein the second time period is a future time based on a sports event date, wherein the first time period is a duration involving a fitness schedule including a set of fitness activities to progress towards the first goal; executing, via a health engine, a heatmap component configured to provide a pictorial heatmap representation of muscle activation during an exercise including: pictorially representing, at the GUI of the computing device, a first set of muscles of the user engaging in an exercise that should be activated during the exercise using a first color, partially based on a user-specific physical characteristic from the health questionnaire; pictorially representing, at the GUI of the computing device, a second set of muscles of the user engaging in the exercise that should not be activated during the exercise using a second color, partially based on the user-specific physical characteristic from the health questionnaire; adapting the pictorial heatmap representation of muscle activation based on the user-specific physical characteristic, such that the pictorial heatmap representation is affected by the health questionnaire, wherein changing the exercise based on the location of the pain points if the pain points are located within an area of the second color of the pictorial representation; changing the exercise displayed on the GUI if the pain points are located within an area of the second color of the pictorial representation, based on the health engine determining that a current exercise is contraindicated; detecting, by the health engine, user activity data from the wireless health device in real time during exercise performance; dynamically updating, via the health engine, the pictorial heatmap representation as the health engine detects user engagement with the exercise based on the real-time user activity data to provide real-time data based on the adapted pictorial heatmap representation; receiving, by a form detection engine, a real-time video stream of a user performing a physical exercise, wherein the real-time video stream is captured by a client device; analyzing, by the form detection engine, a plurality of sequential video frames from the real-time video stream using a pose estimation algorithm to extract skeletal key points of the user; determining, based on the extracted skeletal key points, an exercise type being performed by the user and calculating joint angles and angular velocities over time; identifying, based on the calculated joint angles and angular velocities, one or more deviations between movement of a user and a reference biomechanical template corresponding to the exercise type; generating, by the form detection engine, a form deviation heatmap that visually depicts the one or more deviations on a human body outline, wherein a color-coded overlay is used to represent one or more of overactive, underactive, or misaligned muscle groups associated with the one or more deviations; and displaying, on a graphical user interface of the client device, the form deviation heatmap with corresponding muscle group indicators and real-time video or animation feedback; generating, by the health engine, a health profile including the health data, the first health goal, and the second health goal for the user; implementing, using an algorithm of the health engine, incorporating the health data in if-then scenarios to determine a first wellness action for the user to complete during the first time period in conjunction with the fitness schedule to achieve the first health goal, and wherein the first wellness action is a personalized education item that includes at least one of text, graphics, videos, and media, wherein the first wellness action based on the first health goal, and a second wellness action for the user to complete prior to the sports event date, and wherein the algorithm of the health engine is based on a hierarchy of skill development functions having a first level including one or more foundational elements, wherein the hierarchy has a logical order that builds upon the foundational elements and progresses to finer points at a second and third level of the hierarchy to form habits consistent with the first health goal and the second health goals; wherein the algorithm further evaluates the real-time biometric data to detect deviations from expected training intensity and adjusts the second wellness action based on the deviations; and creating, by the health engine, a health and wellness program in the health profile based on the first wellness action and the second wellness action.

In some aspects, the techniques described herein relate to a method, wherein the health data is selected from the group consisting of: medical data, genetic data, nutritional data, fitness data, and environmental data.

In some aspects, the techniques described herein relate to a method, wherein the medical data is selected from the group consisting of: a known health problem of the user, a prior health problem of the user, a current health problem of the user, a health problem of a family member associated with the user, and a physiological or biochemical measurement of the user.

In some aspects, the techniques described herein relate to a method, wherein the physiological or biochemical measurement of the user is selected from the group consisting of: a heart rate measurement, a resting metabolic rate (RMR) measurement, an oxygen consumption (V02) level measurement, a weight measurement, a body fat measurement, a visceral fat measurement, a muscle mass measurement, a measurement of body water of the user, a body mass index (BMI) measurement, a bone mass measurement, and a blood glucose level measurement.

In some aspects, the techniques described herein relate to a method, wherein the genetic data includes genomic information.

In some aspects, the techniques described herein relate to a method, wherein the fitness data is selected from the group consisting of: a type of exercise routine engaged in by the user, a type of workout engaged in by the user, a length of time spent on the exercise routine, a length of time spent on the workout a number of calories burned during the exercise routine, a number of calories burned during the workout, a heart rate achieved during the exercise routine, and a heart rate achieved during the workout.

In some aspects, the techniques described herein relate to a method, wherein the environmental data includes a lifestyle choice of the user, and wherein the lifestyle choice of the user is selected from the group consisting of: a sleep habit of the user, a type of learner the user is, a smoking habit of the user, and an alcohol intake habit of the user.

In some aspects, the techniques described herein relate to a method, wherein the nutritional data includes information selected from the group consisting of: types of foods eaten by the user, a number of daily calories consumed by the user, a quantity of meals consumed daily by the user, a quantity of snacks consumed daily by the user, a type of snacks consumed daily by the user, a type of beverage consumed daily by the user, and a quantity of beverages consumed daily by the user.

In some aspects, the techniques described herein relate to a method, wherein the first time period is a current time period, and wherein the second time period is a future time period.

In some aspects, the techniques described herein relate to a method, wherein the health data pertaining to the user is received from one or more wireless health devices tracking one or more biometric parameters of the user.

In some aspects, the techniques described herein relate to a method, further including dynamically updating the form deviation heatmap in real time as the user performs additional repetitions of the physical exercise, such that the displayed muscle group indicators reflect evolving deviations.

In some aspects, the techniques described herein relate to a method, further including modifying the reference biomechanical template based on body-type metadata obtained from a pre-exercise intake questionnaire completed by the user.

In some aspects, the techniques described herein relate to a method, further including detecting one or more specific deviation patterns from the exercise type and, in response, generating a corrective exercise recommendation selected from a predetermined list of mobility or activation drills.

In some aspects, the techniques described herein relate to a method, wherein the corrective exercise recommendation is presented to the user in the form of an in-application message or as an adjustment to a workout schedule displayed by the graphical user interface, wherein the reference biomechanical template is selected based on one or more user-specific characteristics including limb length ratios, historical injury data, or fitness level, and wherein the pose estimation algorithm includes a convolutional neural network trained on labeled video datasets containing annotated joint trajectories.

In some aspects, the techniques described herein relate to a method, further including calculating a confidence score for each of the one or more deviations, and suppressing visual indicators on the form deviation heatmap below a predefined threshold.

In some aspects, the techniques described herein relate to a method, wherein the pose estimation algorithm includes a spatial-temporal model that leverages optical flow between consecutive video frames to improve key point tracking fidelity, wherein the color-coded overlay within the form deviation heatmap includes a muscle activation region rendered in accordance with inferred electromyographic activity data derived from a joint motion pattern of a user.

In some aspects, the techniques described herein relate to a method, further including receiving real-time biometric data from a wearable sensor worn by the user, and correlating the real-time biometric data with pose-derived metrics to refine accuracy of the form deviation heatmap, wherein the real-time biometric data includes heart rate, skin temperature, or an electromyography signal.

In some aspects, the techniques described herein relate to a method, further including classifying the physical exercise as belonging to one of a plurality of predefined exercise families using a video classifier engine, wherein the video classifier engine includes temporal convolution layers and attention layers configured to improve classification accuracy for compound movement exercises.

In some aspects, the techniques described herein relate to a method, wherein the graphical user interface enables the user to enter subjective feedback data regarding a rating of perceived exertion for the physical exercise, and wherein the subjective feedback data is used to adjust a level of difficulty of one or more subsequent exercises in a workout program, and wherein the color-coded overlay in the form deviation heatmap includes a red gradient to indicate overactive muscle groups, a blue gradient to indicate underactive muscle groups, and a green region to indicate proper alignment.

In some aspects, the techniques described herein relate to a method, further including: storing historical deviation data associated with the user in a database and training a personalization model to anticipate future form breakdowns based on indicators of fatigue or workout duration, and wherein the personalization model is configured to generate one or more real-time prompts instructing the user to pause or modify the physical exercise based on a predicted injury risk; and generating an assessment report that includes the form deviation heatmap, the one or more deviations, one or more corrective action recommendations, and links to one or more video tutorials.

### BRIEF DESCRIPTION OF THE DRAWINGS

The present disclosure may be better understood, and its numerous features and advantages made apparent to those skilled in the art, by referencing the accompanying drawings. The use of the same reference symbols in different drawings indicates similar or identical items.

FIG. 1-FIG. 2 depict perspective views of a computer system configured to implement a method for real-time fitness tracking and scheduling, according to at least some embodiments described herein.

FIG. 3 depicts a graphical representation of an algorithm configured to implement a method for real-time fitness tracking and scheduling, according to at least some embodiments described herein.

FIG. 4 depicts a perspective view of an intake screen of a health engine associated with a computer system configured to implement a method for real-time long-term and short-term fitness and/or health/wellness tracking and scheduling, according to at least some embodiments described herein.

FIG. 5-FIG. 6 depict perspective views of a medical history screen of a health engine, according to at least some embodiments described herein.

FIG. 7 depicts a perspective view of pain points associated with a medical history screen of a health engine, according to at least some embodiments described herein.

FIG. 8 depicts a perspective view of a login screen associated with a health engine, according to at least some embodiments described herein.

FIG. 9-FIG. 10 depict perspective views of a health profile associated with a health engine, according to at least some embodiments described herein.

FIG. 11-FIG. 13 depict perspective views of an equipment screen associated with a health engine, according to at least some embodiments described herein.

FIG. 14 depicts a perspective view of a health profile associated with a health engine, where the health profile depicts a workout session, according to at least some embodiments 20 described herein.

FIG. 15-FIG. 16 depict perspective views of a representation of an individual exercise associated with a workout session of a health profile, according to at least some embodiments described herein.

FIG. 17 depicts a perspective view of a nutrition module associated with a health profile, according to at least some embodiments described herein.

FIG. 18-FIG. 19 depict perspective views of individual exercises of a workout session associated with a health profile, according to at least some embodiments described herein.

FIG. 20-FIG. 21 depict perspective views of an alert displayed via a health profile, according to at least some embodiments described herein.

FIG. 22 is a block diagram of a computing device included within the computer system of FIG. 1 and/or FIG. 2 that is configured for real-time fitness tracking and scheduling, in accordance with embodiments of the present invention.

FIG. 23 is an illustration of a device with a display and a graphical user interface (GUI) showing an operational sequence that may be performed when running an AI Trainer Application, according to some embodiments.

FIG. 24 is an illustration of a device with a display and a graphical user interface (GUI) showing an operational sequence that may be performed when running an AI Trainer Application, according to some embodiments.

FIG. 25 is an illustration of a device with a display and a graphical user interface (GUI) showing an operational sequence that may be performed when running an AI Trainer Application, according to some embodiments.

FIG. 26 is an illustration of a device with a display and a graphical user interface (GUI) showing an operational sequence that may be performed when running an AI Trainer Application, according to some embodiments. FIG. 27 is an illustration of a device with a display and a graphical user interface (GUI) showing an operational sequence that may be performed when running an AI Trainer Application, according to some embodiments.

FIG. 28 is an illustration of a device with a display and a graphical user interface (GUI) showing an operational sequence that may be performed when running an AI Trainer Application, according to some embodiments.

FIG. 29 is an illustration of a device with a display and a graphical user interface (GUI) showing an operational sequence that may be performed when running an AI Trainer Application, according to some embodiments.

FIG. 30 is an illustration of a device with a display and a graphical user interface (GUI) showing an operational sequence that may be performed when running an AI Trainer Application, according to some embodiments.

### DETAILED DESCRIPTION

The preferred embodiments of the present invention will now be described with reference to the drawings. Identical elements in the various figures are identified with the same reference numerals.

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

Described herein is a method and system to create a user-specific and personalized health and wellness program. The instant invention provides individualized steps that create a truly personalized, stepwise approach or program that unfolds/evolves as the user participates over time.

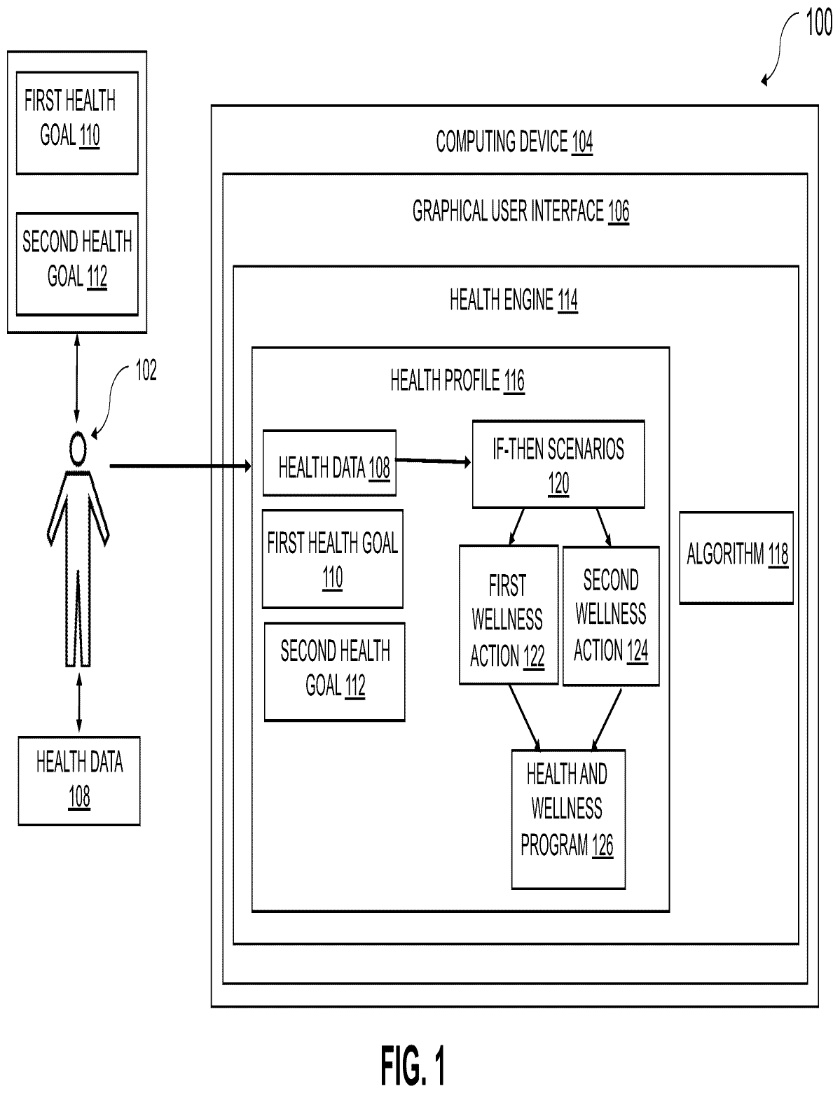

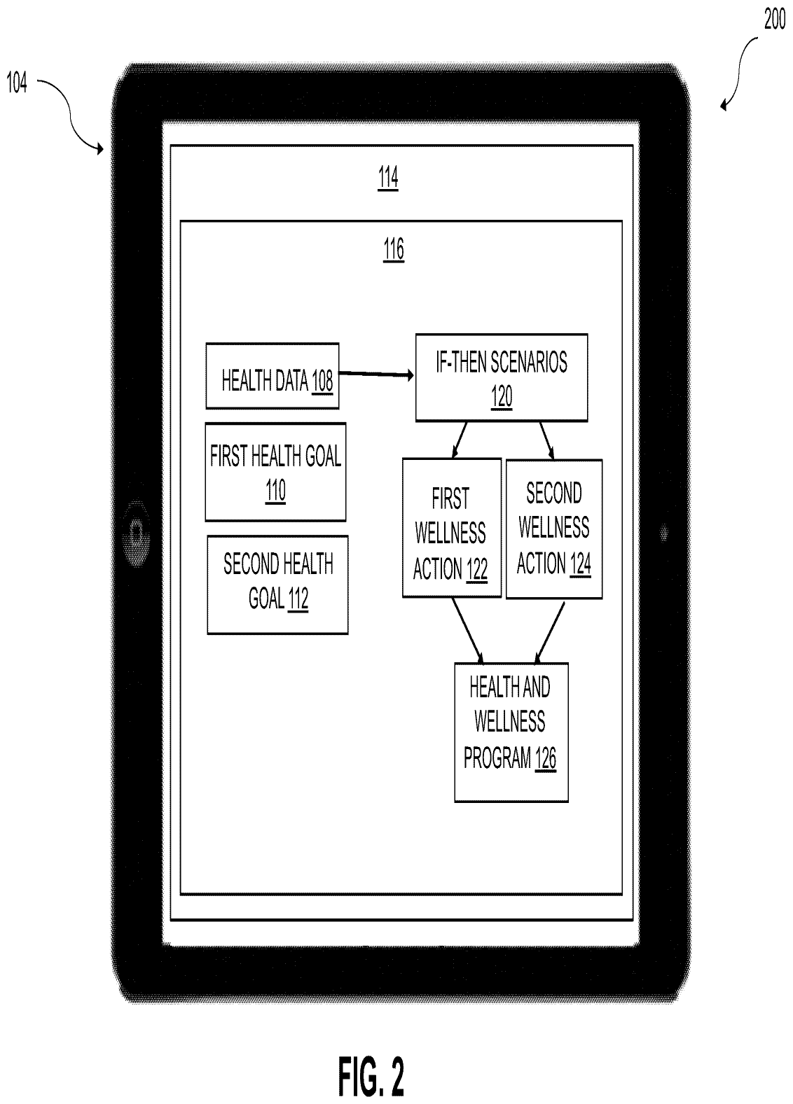

FIG. 1 and FIG. 2 depict a computer system 100 and a computer system 200, respectively, configured to implement a method for real-time fitness tracking and scheduling Specifically, the computer system 100 and the computer system 200, respectively, implement the method for the real-time fitness tracking and scheduling, which includes providing both a macroanalysis and a micro-analysis of health data of a user, assessing the health data in if-then scenarios to determine user-specific wellness actions for a user to complete to achieve user specific health goals, and generating a health and wellness program based on these user-specific wellness actions to achieve the user-specific health goals. Specifically, the micro-analysis includes decisions that guide the macro-analysis based on the user-specific health goals and needs. Moreover, the method for the real-time fitness tracking and scheduling identifies user health priorities and establishes a user-directed fitness pacing system that is both flexible and responsive to a user 102. The user-directed fitness pacing system provides long-term fitness management for the user 102.

The computer system 100 may include a computing device 104. The computing device 104 may be a computer, a laptop computer, a smartphone, and/or a tablet, among other examples not explicitly listed herein. The computing device 104 may comprise a health engine 114 that may execute the method for real-time fitness tracking and scheduling. In other examples, the health engine 114 may be a health application, a health software program, a health service, or a health software platform configured to be executable on the computing device 104. The user 102 may interact directly with the health engine 114 via a graphical user interface (GUI) 106 of the computing device 104.

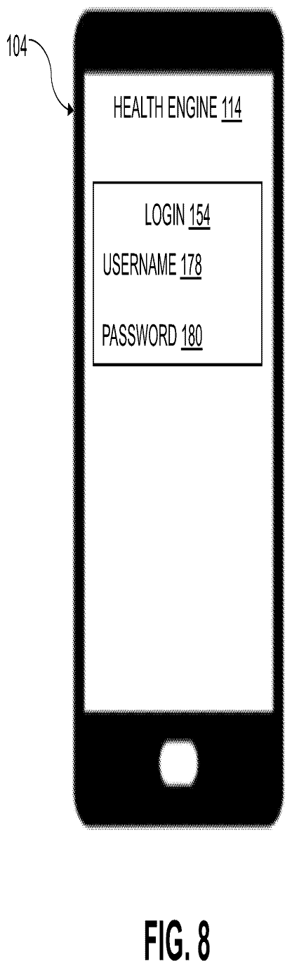

As shown in FIG. 8, the health engine 114 may receive, from the user 102, login credentials such that the user 102 may login 154 to and interact with the health engine 114. The login credentials may include a username 178, a password 180, a biometric identification means (e.g., fingerprint identification, face recognition identification, palm print identification, iris recognition, retina recognition, etc.), etc. In response, the health engine 114 identifies the user 102 based on the login credentials.

Identification of the user 102 may include information such as: a name of the user 102, telephone number of the user 102, an address of the user 102, etc. In some examples, identifying the user 102 based on the login credentials may include determining that the user 102 has a health profile 116 associated with the health engine 114. If it is determined that the user 102 does not yet have the health profile 116, the health engine 114 may prompt the user 102 to create such profile. Responsive to creation of such profile, the health engine 114 may grant the user 102 access to the health engine 114.

Further, the health engine 114 may receive health data 108 pertaining to the user 102. The health data 108 may be received by the health engine 114 from one or more wired or wireless health devices tracking one or more biometric parameters of the user 102. The one or more wired or wireless health devices may continuously track and update the one or more biometric parameters of the user 102. Examples of these wired or wireless health devices include: watches, bracelets, wristbands, ankle bands, rings, or necklaces, among other examples not explicitly listed herein. Examples of the one or more biometric parameters of the user 102 include: a heartrate of the user 160, a quantity of calories burned by the user 160, and/or a blood pressure of the user 160, among other parameters not explicitly listed herein.

In other examples, the health data 108 pertaining to the user 102 may be received from user input (e.g., audio or textual) via the GUI 106 in response to the health questionnaire (e.g., the intake questionnaire 134). The health engine 114 may also receive a first health goal 110 and/or a second health goal 112 of the user 102 from the user input via the GUI 106 in response to the health questionnaire.

The health questionnaire may include questions, such as, “what time do you awaken in the morning?”; “what time do you exercise?”; “how many times do you eat each day?”; “what time is your first meal?”; “what are the contents of your first meal?”; “what time is your second meal?”; “what are the contents of your second meal?”; “what time is your third meal?”; “what are the contents of your third meal?”; “what time do you go to sleep?”; “how well do you sleep?”; “do you have any past injuries or surgeries?”; “do you have any current or chronic musculoskeletal pain or ailments?”; “do you work out or exercise?”; “how often do you work out or exercise?”; “do you enjoy working out?”; “have you attempted to meet any health or fitness goals before?”; “have you met any health or fitness goals before?”; “are you training to improve in a specific sport?”, etc. It should be appreciated that the listed questions are for illustrative purposes only and the questions are not limited to those explicitly listed herein. Moreover, if the user 102 is training to improve in a specific sport, such as body building, cycling, running, baseball, basketball, tennis, football, etc., the health questionnaire may additionally include the following questions, “have you ever experienced a fracture?”; “do you have any chronic tissue injuries?”; “do you have any joint pain?”, etc.

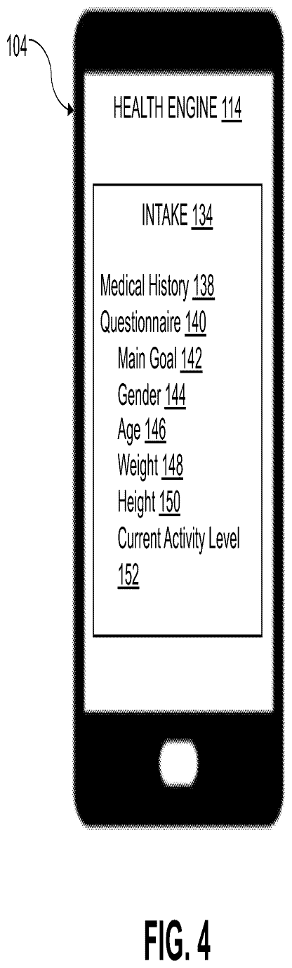

More specifically, as shown in FIG. 4, questions 140 of the intake questionnaire 134 may be based on: a medical history 138 of the user 102, health/wellness goals 142 the user 102 wishes 20 to accomplish (e.g., run a half-marathon), a gender 144 of the user 102, an age 146 of the user 102, a weight 148 of the user 102, a height 150 of the user 102, a current activity level 152 of the user 102, etc. In some examples, the intake questionnaire 134 is a mosaic questionnaire. In the mosaic questionnaire, a portion of the questions may not be filled out from the beginning by the 16 5 10 15 20 user 102. In other examples, the questions 140 of the intake questionnaire 134 change/evolve based on the input received from the user 102.

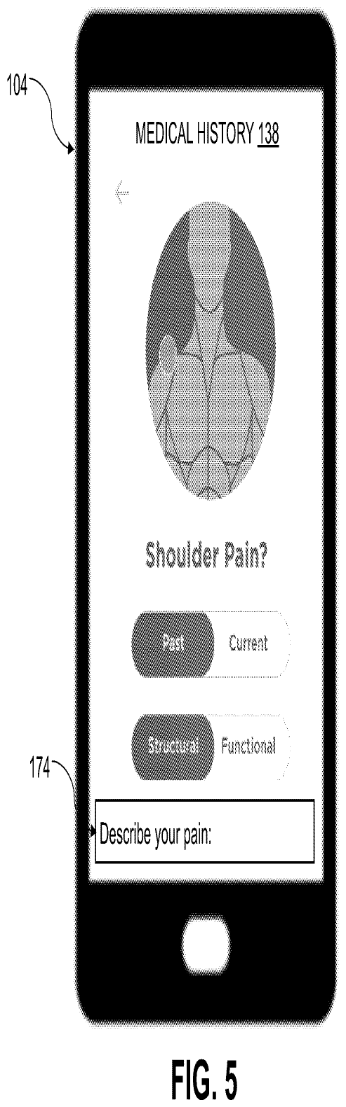

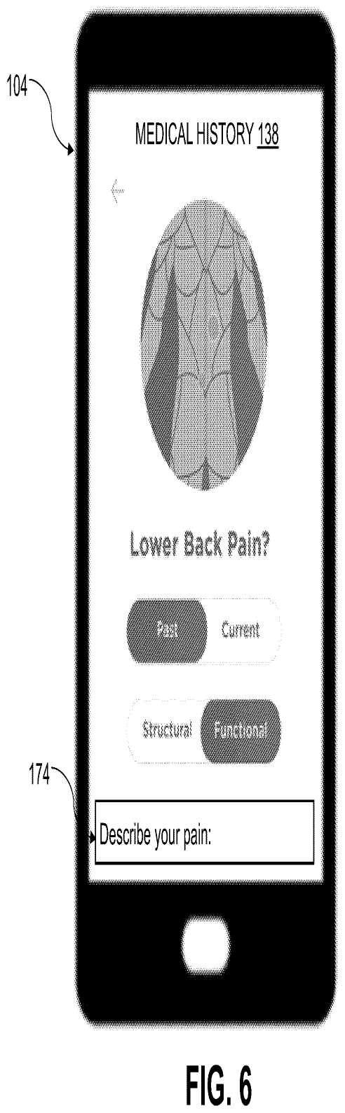

In some examples, the user 102 may indicate or input into the health engine 114 that the user 102 has or has had some sort of pain (such as shoulder pain, lower back pain, leg pain, etc.). In response, the health engine 114 may display a graphic of such pain, as shown in FIG. 5 and FIG. 6. Moreover, the health engine may prompt the user 102 to select whether such pain is past or current, whether such pain is structural or functional, etc. Moreover, the health engine 114 may prompt the user 102 to describe the pain in a text entry box 174. Such textual description is subsequently analyzed by the health engine 114.

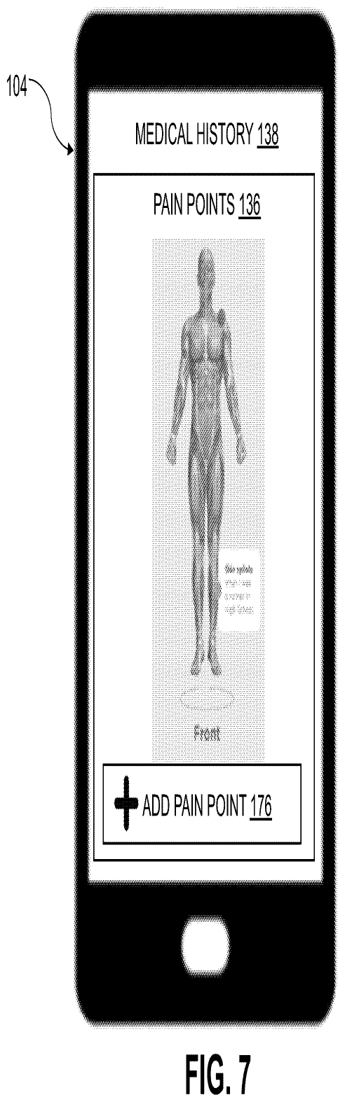

In other examples, and as shown in FIG. 7, the health engine 114 may prompt the user 102 to select pain points 136 on a graphical representation of a human body. The user 102 may also add pain points 176 to the graphical representation of the human body. All of the gathered health data 108 may be used/analyzed by the health engine 114.

The first health goal 110 of the user 102 may be configured for completion during a first time period and the second health goal 112 of the user 102 may be configured for completion during a second time period. In some examples, the first time period is a current time period and the second time period is a future time period. In other examples, the second time period is the current time period and the first time period is the future time period. As an illustrative example, the first health goal 110 for completion during the current time period may include losing body fat and the second health goal 112 for completion during the future time period may include running a marathon. Other examples of the first health goal 110 and/or the second health goal 112 may include: losing weight, improving body composition or tone, building muscle, getting stronger or more powerful, sport specific goals, relieving pain, etc.

In examples, the health data 108 described may include medical data, genetic data, nutritional data, fitness data, and/or environmental data, among other data not explicitly listed herein. In some examples, the health data may additionally include stories of the user that encompass the medical data, the genetic data, subjective measures (e.g., self-reported by the 5 user), the nutritional data, the fitness data, and/or the environmental data. In some examples, the medical data may prove to be important, as it may indicate risk factors for the user 102 for chronic diseases, such as hypertension, hypercholesterolemia, coronary artery disease, cancer, and/or diabetes, etc. Examples of the medical data may include: a known health problem of the user 102, a prior health problem of the user 102, a current health problem of the user 102, a 10 health problem of a family member associated with the user 102, and/or a physiological or biochemical measurement of the user 102, among other medical data not explicitly listed herein. The physiological or biochemical measurement of the user 102 may include: a heart rate measurement, a resting metabolic rate (RMR) measurement, an oxygen consumption (VO2) level measurement, a weight measurement, a body fat measurement, a visceral fat measurement, a muscle mass measurement, a measurement of body water of the user 102, a body mass index (BMI) measurement, a bone mass measurement, and/or a blood glucose level measurement, among other measurements not explicitly listed herein.

In other examples, the genetic data may include genomic information associated with the user 102. In some examples, the genomic information may be targeted and may be specifically related to genetic correlations with diseases. In other examples, the nutritional data may include information such as: types of foods eaten by the user 102, a number of daily calories consumed by the user 102, a quantity of meals consumed daily by the user 102, a quantity of snacks consumed daily by the user 102, a type of snacks consumed daily by the user 102, a type of beverage consumed daily by the user 102, and/or a quantity of beverages consumed daily by the user 102, among other data not explicitly listed herein. It should be appreciated that more questions may be asked than what is visually represented to gain a better understanding of the pain points of the user 102 and how to work with those pain points (e.g., either how to change exercises, avoid exercises, or maybe even suggest contacting a medical professional before returning to exercise).

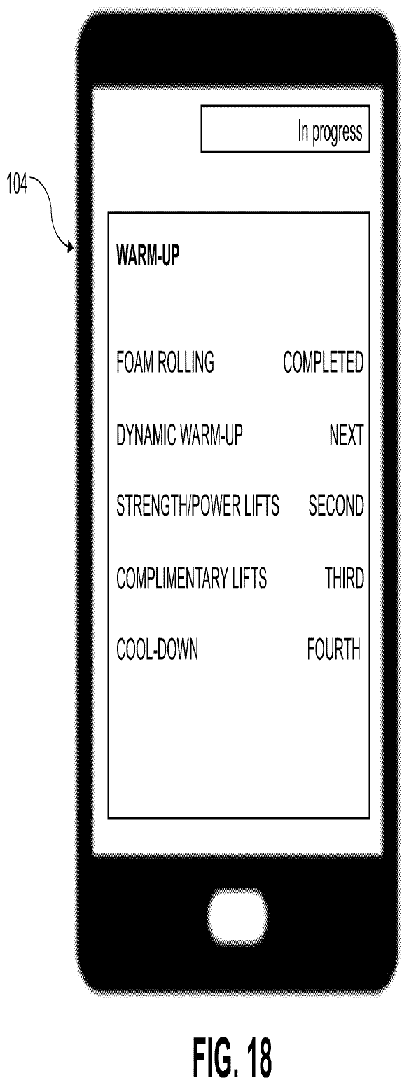

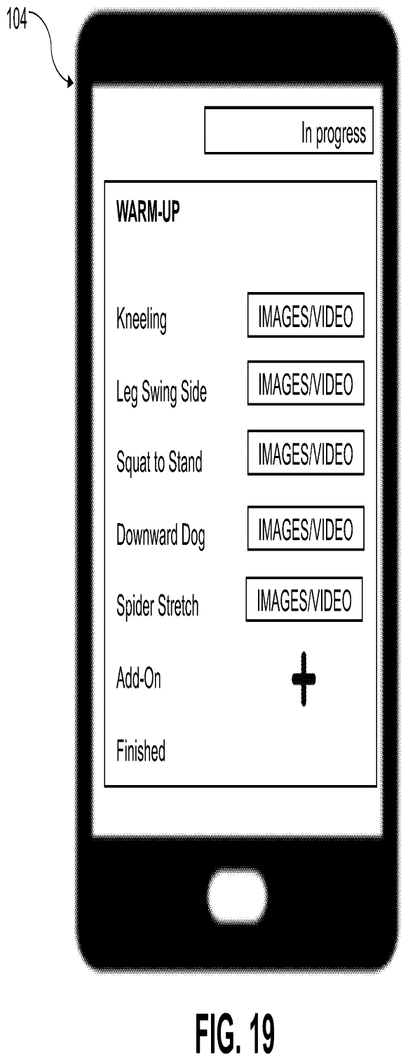

In additional examples, fitness data (as shown in FIG. 18 and FIG. 19) may include: a type of exercise routine engaged in by the user 102, a type of workout engaged in by the user 102, a length of time spent on the exercise routine, a length of time spent on the workout a number of calories burned during the exercise routine, a number of calories burned during the workout, a heart rate achieved during the exercise routine, and/or a heart rate achieved during the workout, among other data not explicitly listed herein. As depicted in FIG. 18 and FIG. 19, the fitness data includes multiple exercises to be completed by the user 102, such as a spider stretch, and a downward dog stretch. As depicted, the user 102 can also view images of the exercises 15 directly on the GUI.

Moreover, examples of the environmental data may include one or more lifestyle choices of the user 102. In examples, the one or more lifestyle choices of the user 102 may include: a sleep habit of the user 102, a type of learner the user 102 is (e.g., a visual learner, an auditory learner, or a heuristic learner), a smoking habit of the user 102, and/or an alcohol intake habit of the user 102, among other data not explicitly listed herein.

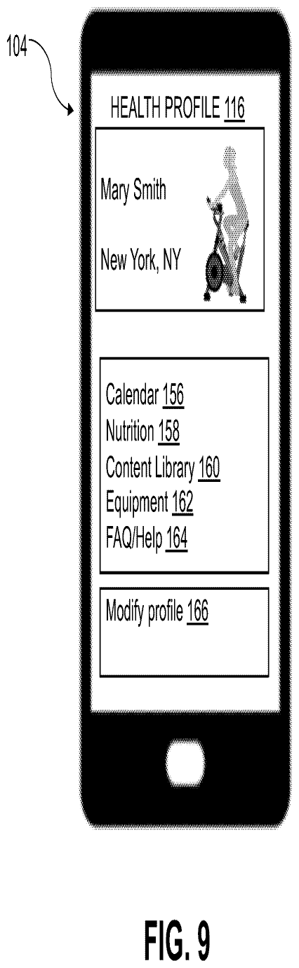

Then, the health engine 114 may create a health profile 116 for the user 102 that includes the health data 108, the first health goal 110, and/or the second health goal 112, among other information. The health profile 116 may also include information about the user, such as a name, 19 5 10 15 an address, a photograph, a graphic, a bit Moji, etc. As shown in FIG. 9, the health profile 116 may include one or more modules, such as a calendar module 156, a nutrition module 158, a content library 160, an equipment module 162, and/or a frequently asked questions (FAQ)/help module 164, among others not explicitly listed herein.

The user 102 may input exercises performed by the user 102 into the calendar module 156. The calendar module 156 may distinguish between different types of exercises by using unique colors, text, font, etc. for each exercise. The calendar module 156 may also be used to track food items or meals eaten by the user 102. The content library 160 may include users elected infographics, videos, images, books, articles, etc. focused around health/wellness.

The user 102 may also engage in actions to modify 166 the health profile 116, which may include adding content to the health profile 116, deleting content from the health profile 116, and/or cancelling or deleting the health profile 116. The user 102 may also engage in actions to upload the users 102 own picture/photograph or modify an existing picture/photograph.

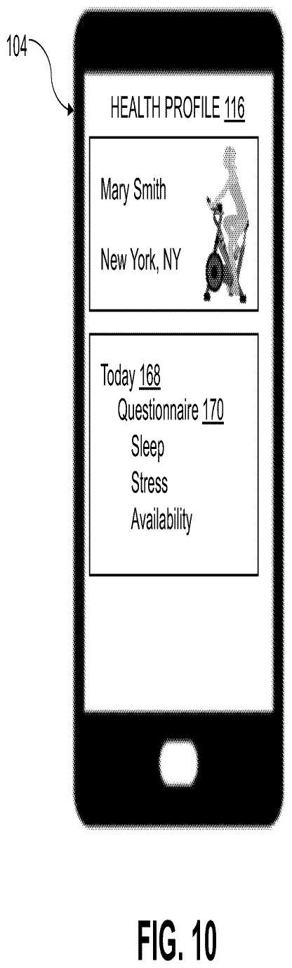

As shown in FIG. 10, the health profile 116 may include a today module 168. The today module 168 may include a questionnaire 170 regarding how the user 102 is feeling on a given day. The questionnaire 170 may pose questions to the user 102, such as an amount of sleep the user 102 got the previous night, a stress level of the user 102, an availability of the user 102, a current energy level of the user, etc. In response to each of the questions of the questionnaire 170, the user 102 may select a specific number (e.g., between 1-20), a quantity of an object (e.g., 20 4/5 stars), etc.

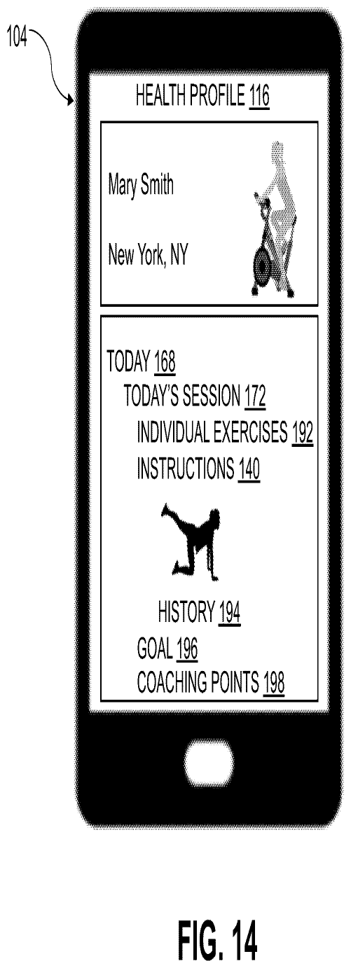

As shown in FIG. 14, the today module 168 may also include information regarding: today's workout session 172, individual exercises 192 within today's workout session 172, instructions on how to perform each of the individual exercises 192, a history 194 regarding the 20 5 10 15 20 user 102 performing each of the individual exercises 192 and/or the workout session 172, current fitness goals 196 of the user 102, and/or coaching points 198 for the user 102. The user 102 also has the ability to “scramble” or switch up today's workout session 172 or each of the individual exercises 192 within today's workout session 172 based on the users preferences.

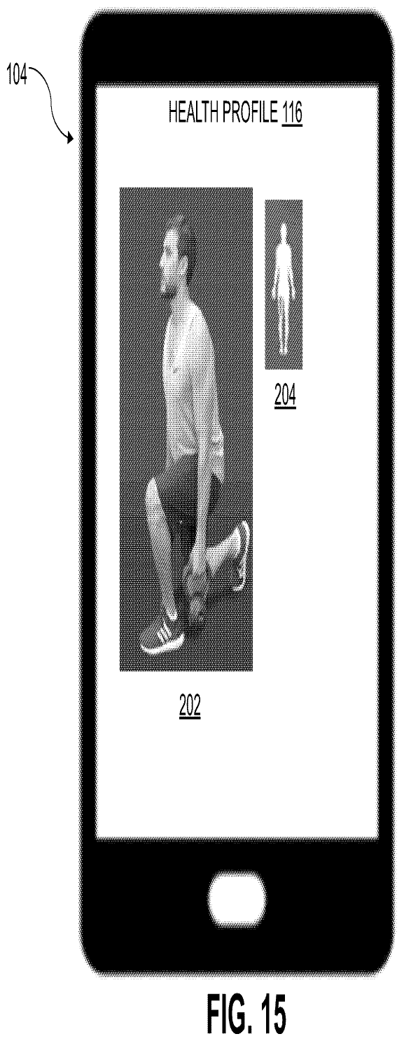

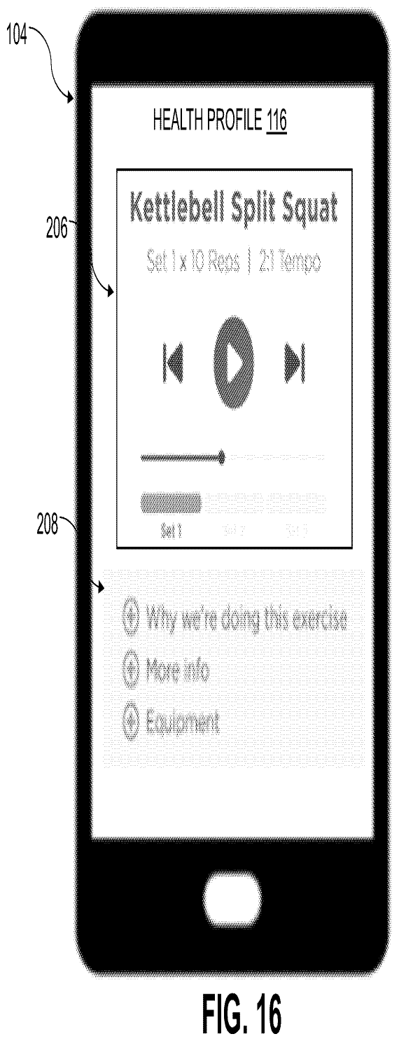

The instructions on how to perform each of the individual exercises 192 within today's workout session 172 may be textual, audio, or graphical (e.g., an image or video). FIG. 15 shows a graphical representation 202 of an individual exercise 192 within today's workout session 172. Moreover, FIG. 15 shows a graphical representation 204 of a body part that each of the individual exercises 192 targets. The instructions on how to perform each of the individual exercises 192 may also include additional information 208 of FIG. 16 regarding why the user 102 is performing this exercise, more information about the exercise, and equipment needed to perform the given exercise.

In some examples, the instructions on how to perform each individual exercises 192 may include a trainer performing such exercise in real-time. Moreover, the coaching points 198 of FIG. 14 may include textual, audio, or graphical (e.g., an image or video) instructions to the user 102 on how to perform each of the individual exercises 192 within today's workout session 172 more accurately.

Moreover, in some implementations, a heatmap component (not shown) may be incorporated into this invention. At any given point while the user 102 is engaging with a given exercise, the heatmap component (executed by the health engine 114) pictorially depicts which muscles should be working. The muscles that should be working may be shown in red, whereas the muscles that should not be working may be shown in blue. Such colors are provided for illustrative purposes only and other colors are contemplated. Such heatmap component may also be affected by the intake questionnaire 134. For example, if the user 102 specifies in the intake questionnaire 134 that the user 102 is short-limbed and long-torsoed, the heatmap component may differ than if the user 102 specifies in the intake questionnaire 134 that the user 102 is short torsoed and long-limbed.

In some embodiments, the health engine may include or be in communication with a heatmap generation subsystem configured to dynamically update a pictorial representation of muscle activation while a user is engaging in an exercise. The heatmap subsystem may receive real-time data from one or more wearable sensors, cameras, or other input devices associated with the computing device. The wearable sensors may include accelerometers, gyroscopes, magnetometers, and inertial measurement units (IMUs) configured to detect user movement patterns, orientation, velocity, and range of motion. Additional biometric sensors, such as heart rate monitors, surface electromyography (sEMG) sensors, and strain or pressure sensors embedded in garments, may provide physiological data indicating muscular activation, effort, or contraction duration.

The health engine processes the collected data using an exercise recognition module that classifies the user's current activity. This module applies pattern recognition and motion classification algorithms, which may include trained machine learning models that match sensor-derived motion sequences to known exercise templates stored in an exercise database. Each stored exercise template includes a biomechanical profile specifying expected joint angles, segment velocities, and muscle activation sequences associated with that exercise. For instance, a database entry for a squat may include characteristic motion trajectories for the hip, knee, and ankle joints and an associated activation map identifying the gluteus maximus, quadriceps, and hamstring muscle groups as primary movers.

When the health engine determines that a user's motion pattern matches a known exercise, it retrieves the corresponding biomechanical profile and activates a heatmap rendering module. The rendering module generates or updates a pictorial representation of the human body in which each muscle region is represented by a color gradient corresponding to a level of activation. For example, primary muscles engaged in the detected exercise may be rendered in red, secondary stabilizing muscles in orange or yellow, and non-engaged or inactive regions in blue or gray. The heatmap dynamically updates in real time as new data packets are received from the wearable sensors or other sources. The color mapping may shift continuously to reflect changing engagement intensity, thereby providing the user with visual feedback during the exercise.

In certain embodiments, the health engine integrates input from the device's optical sensors or an external camera. The video feed may be processed by a computer vision algorithm that detects skeletal landmarks, joint angles, and posture alignment. Using pose estimation, the system can verify whether the user's current form conforms to the expected template for the detected exercise. Deviations from the standard motion path, such as excessive knee displacement or spinal flexion, may be detected and mapped to specific corrective indicators. The heatmap may then adaptively highlight incorrect or compensatory muscle activations in contrasting colors (e.g., red for overactivation, blue for under activation) to instruct the user visually on form correction.

The health engine fuses these multimodal data inputs through a sensor fusion layer that synchronizes motion, position, and biometric data streams. The fused dataset is processed by an activation inference model that estimates muscle engagement levels based on combined movement and physiological cues. The resulting activation data is transmitted to a graphical rendering engine that executes the heatmap display within the graphical user interface (GUI). The GUI may include controls that allow the user to toggle between static snapshots of muscle engagement and continuously updating live visualizations.

Furthermore, the dynamic heatmap output may be modulated by the user's intake questionnaire data. For example, anthropometric characteristics entered during onboarding, such as limb-to-torso ratio, body composition, and flexibility range, may modify the baseline activation models to account for biomechanical differences. A user who reports having short limbs and a long torso may have a distinct predicted activation profile for the same exercise compared to a user with long limbs and a short torso. The health engine uses these intake parameters to adjust scaling factors within the activation inference model, ensuring that the heatmap more accurately reflects the user's individual biomechanics.

The data pipeline for the dynamic heatmap may thus include: (1) acquisition of movement and biometric data via wearable sensors and/or cameras; (2) classification of the performed exercise via motion recognition algorithms; (3) extraction of expected muscle activation profiles from a stored exercise database; (4) real-time adjustment of activation intensity based on live sensor feedback; (5) dynamic rendering of an adaptive color-coded heatmap within the GUI; and (6) optional correction overlays highlighting improper form or compensatory muscle activation. Through this integration, the health engine is capable of determining which exercise the user is performing and which muscle groups are being activated without manual input. The combination of exercise recognition, biomechanical modeling, and multimodal data fusion enables the health engine to generate and continuously update the heatmap in real time, providing the user with immediate and personalized feedback during physical activity.

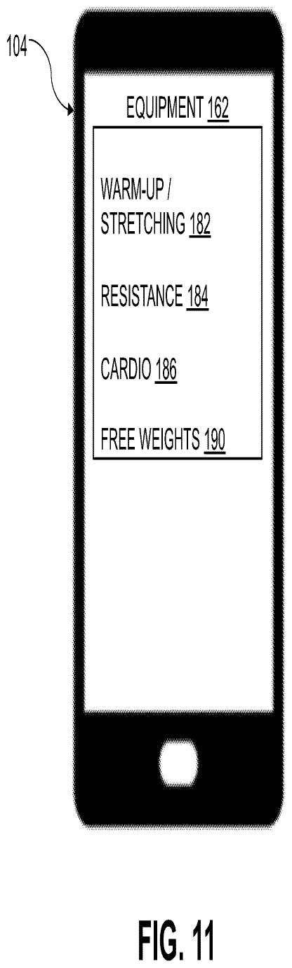

As shown in FIG. 11, the equipment module 162 of the health profile 116 may include categories, such as: warm-up/stretching equipment 182, resistance equipment 184, cardio equipment 186, and/or free weights equipment 190, among others not explicitly listed herein. Each of these categories may include common equipment used for the given category and a list of suggested equipment for purchase 188. The suggested equipment for purchase 188 may include graphics of the suggested equipment, website links to places the user 102 can purchase the suggested equipment, prices of the suggested equipment, physical locations/stores where the user 102 may purchase the suggested equipment, etc. The user 102 may also filter the suggested equipment for purchase 188 based on the users 102 fitness goals, a budget of the user 102, space the user 102 has to dedicate to fitness equipment, etc.

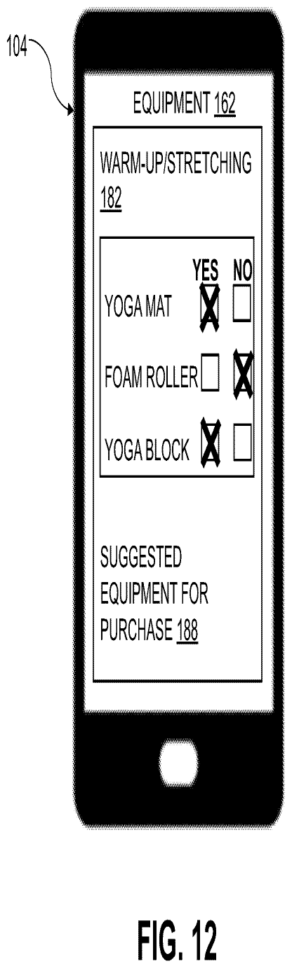

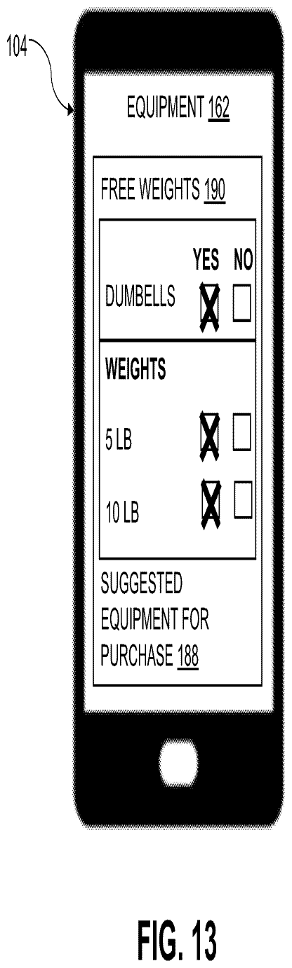

As an illustrative example, the warm-up/stretching equipment 182 category is depicted in more detail in FIG. 12. For example, the warm-up/stretching equipment 182 may include: a yoga mat, a foam roller, and/or a yoga block, among other pieces of equipment not explicitly listed herein. The user 102 may then select which pieces of equipment the user 102 has and/or may browse and/or purchase any of the suggested equipment 188.

As another illustrative example, the free weights category 190 is depicted in more detail in FIG. 13. As an example, the free weights category 190 may include dumbbells of varying weights (e.g., 2 lbs., 5 lbs., 10 lbs., 20 lbs., etc.). The user 102 may then select which pieces of equipment the user 102 has and/or may browse and/or purchase any of the suggested equipment 188.

Next, the health engine 114 may implement or incorporate the health data 108 in if-then scenarios 120 to determine a first wellness action 122 (e.g., a “need”) for the user 102 to complete during the first time period to achieve the first health goal 110 (e.g., a “want” of the user 102) and a second wellness action 124 (e.g., a “need”) for the user 102 to complete during the second time period to achieve the second health goal 112 (e.g., a “want” of the user 102). As an illustrative example, the user 102 may have the first health goal 110 (e.g., the “want”) of toning the user's overall body and the first wellness action 122 (e.g., the “need”) may be the user 102 engaging in high intensity interval training exercise three to four times a week. If the user 102 does not focus heavily on engaging in the high intensity interval training exercise three to four times a week, as suggested, to meet the first health goal 110, the health engine 114 may not face liability from this user-driven decision.

In some examples, the health engine 114 may use an algorithm 118 during the if-then scenarios 120 to assess all of the health data 108 (or factors) contributing to the health of the user 102 to help the user 102 form healthy habits gradually and intentionally until they are automated, internalized, and intuitive to the user 102. In some examples, the algorithm 118 may include an artificial intelligence (AI) algorithm, a deep learning algorithm, a decision-making algorithm, or an AI computer vision algorithm. However, the algorithm 118 is not limited to these examples explicitly listed herein.

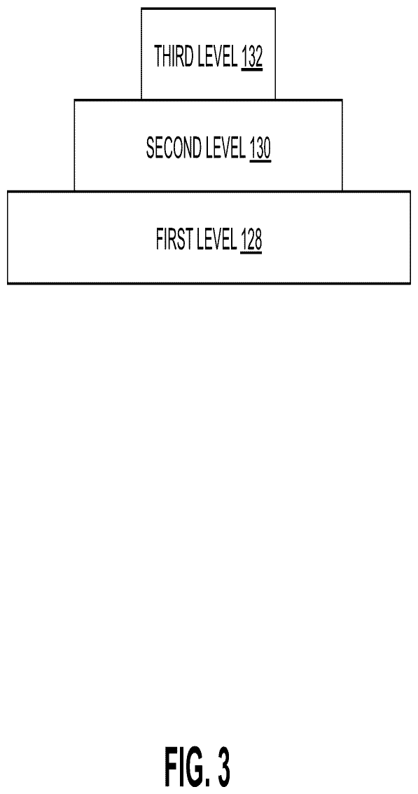

As shown in FIG. 3, the algorithm 118 described herein functions to help the user 102 develop skills that have a logical order and are meant to be built on top of each other to form habits, beginning with more general, foundational elements (e.g., learning the basics of a 23 5 10 15 workout and/or learning basic nutritional principles) (e.g., a first level 128) and moving to finer points (e.g., a second level 130 and a third level 132).

As a first illustrative example, assume that the first health goal 110 for completion during the current time period may include losing body fat. Utilizing the if-then scenarios 120, if the user 102 has a family history of heart disease, then the first wellness action 122 for the user 102 to complete during the first time period to achieve the first health goal 110 of losing body fat may include eating a well-balanced diet. Utilizing the if-then scenarios 120, if the user 102 rarely or never exercises and has no pre-existing injuries, then the first wellness action 122 for the user 102 to complete during the first time period to achieve the first health goal 110 of losing body fat may include eating a well-balanced diet and/or engaging in multiple high intensity interval training sessions. Utilizing the if-then scenarios 120, if the user 102 rarely or never exercises, has no pre-existing injuries, and has met fitness or health goals prior or does not easily get discouraged from trying new things, then the first wellness action 122 for the user 102 to complete during the first time period to achieve the first health goal 110 of losing body fat may include eating a well-balanced diet and/or engaging in exercises new to the user 102. Utilizing the if-then scenarios 120, if the user 102 rarely or never exercises, has no pre-existing injuries, and has not met fitness or health goals prior or easily get discouraged trying new things, then the first wellness action 122 for the user 102 to complete during the first time period to achieve the first health goal 110 of losing body fat may include eating a well-balanced diet and/or engaging 20 in exercises known or common to the user 102.

As another illustrative example, assume that the second health goal 112 for completion during the future time period may include running a marathon. Utilizing the if-then scenarios 120, if the user 102 has the family history of heart disease, then the second wellness action 124 for the user 102 to complete during the second time period to achieve the second health goal 112 of running a marathon may include a workout plan whereby the user 102 continually increases the amount of miles he/she runs on a weekly basis. Utilizing the if-then scenarios 120, if the user 102 rarely or never exercises and has no pre-existing injuries, then the second wellness action 124 for the user 102 to complete during the second time period to achieve the second health goal 112 of running a marathon may include eating a well-balanced diet and/or engaging in weekly running sessions.

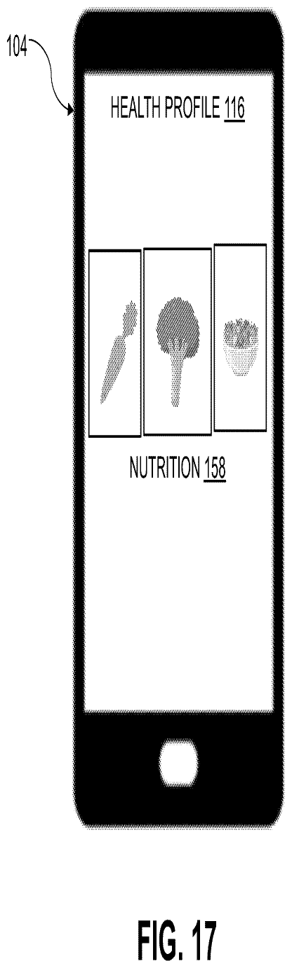

As a further illustrative example, assume that the first health goal 110 for completion during the current time period may include toning one or more muscles of a body of the user 102 and the second health goal 112 for completion during the future time period may include competing in an iron man competition. Part of the health data 108 of the user 102 may include nutritional data (e.g., the user 102 consumes over 2,000 calories daily, with a majority of the calories being present in salty or fatty foods). Furthermore, the user 102 may add or delete food items and/or meals via the nutrition module 158 of FIG. 17.

Utilizing the if-then scenarios 120, if the user 102 is currently consuming over 2,000 calories daily, then the first wellness action 122 for the user 102 to complete during the first time period to achieve the first health goal 110 of toning one or more muscles of the body of the user 102 may include reducing the users 102 caloric intake and replacing some of the salty or fatty foods with high protein food options. Utilizing the if-then scenarios 120, if the user 102 is currently consuming over 2,000 calories daily, then the second wellness action 124 for the user 102 to complete during the second time period to achieve the second health goal 112 of competing in an iron man competition may include reducing the intake of salty or fatty foods 25 5 10 and/or eating a paleo or vegan diet. It should be appreciated that the examples provided herein are non-exhaustive and used for illustrative purposes only.

Then, the health engine 114 may create a health and wellness program 126 in the health profile116 for the user 102 based on the first wellness action 122 and the second wellness action 124. It should be appreciated that the health and wellness program 126 may be displayed to the user 102 via the GUI 106. Moreover, based on the learning style of the user 102, the health and wellness program 126 may be displayed differently to different users. For example, if the user 102 is a identified as a visual learner, the health and wellness program 126 may be displayed in numerous charts or graphs. If the user 102 is identified as an auditory learner, the health and wellness program 126 may be displayed via one or more audio or video files to the user 102.

The health and wellness program 126 may include personalized workouts for the user 102, daily or weekly actions for the user 102, nutritional recommendations for the user 102, equipment recommendations for the user 102, supplement recommendations for the user 102, and/or personalized education for the user 102 to help the user 102 meet the first health goal 110 15 and/or the second health goal 112. The health and wellness program 126 may be presented to the user 102 via one or more of the modules discussed herein. Moreover, the health and wellness program 126 may also include a list of skills acquired by the user 102 over a time period to encourage the user to gain healthy habits.

In some examples, the health engine 114 may receive user activity information pertaining to the user's participation in the health and wellness program 126 (e.g., via user input) and/or may track metrics of user participation in the health and wellness program 126. In response, the health engine 114 may update the health profile 116 based on the user's participation in the health and wellness program 126.

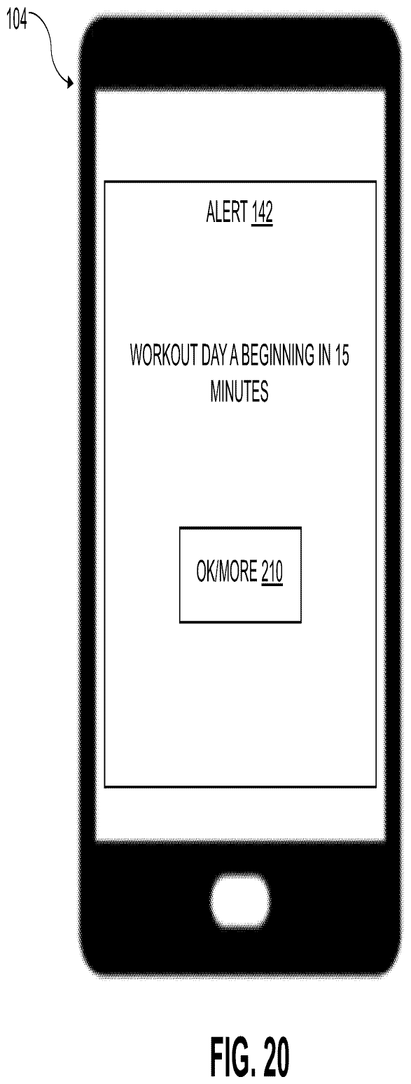

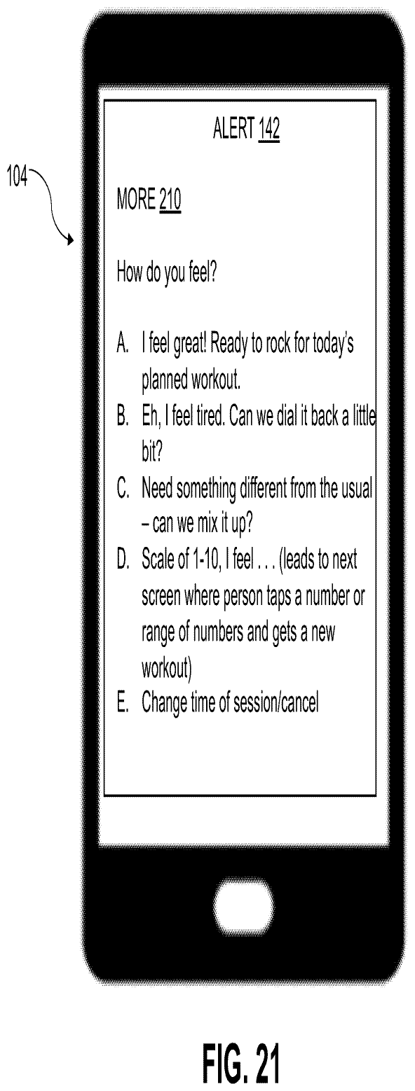

The health engine 114 may also transmit one or more notifications, messages (as depicted in FIG. 21), and/or alerts (as depicted in FIG. 20) to the GUI for display to the user based on the user's participation. The notification may include instructions on how to perform an exercise. As depicted in FIG. 20, the alert may notify or remind the user 102 that a workout or exercise 5 routine should begin in a few minutes. As depicted in FIG. 21, the message may inquire how the user 102 is feeling before or after a particular exercise.

In some examples, the one or more notifications, messages, and/or alerts may include ongoing and personalized education to inform the user 102 on how the user 102 can increase or improve upon his/her performance in the health and wellness program 126. Such ongoing and personalized education may include text, graphics, videos, media, etc. In some examples, the one or more notifications, messages, and/or alerts may include recommendations and/or feedback data to educate, assimilate, and automate the health and wellness program 126 for the user 102. Specifically, the education may include creating user awareness and providing self-directed education based on the user-specific health goals. The assimilation may include turning the 15 education into actionable items (e.g., the first wellness action 122 and/or the second wellness action 124) for the user 102. The automation may include turning the actionable items into habits of the user 102 to create a healthier individual.

In other examples, the one or more notifications, messages, and/or alerts may be generated by a healthcare provider supervising the user's participation in the health and wellness 20 program 126. The healthcare provider may be a nurse, a doctor, a nutritionist, a physical trainer, a fitness coach, etc. In additional examples, the health engine 114 may also provide points and/or rewards to the user 102 based on a level of the user's participation in the health and wellness program 126. In further examples, the health engine 114 may also provide points and rewards to the user 102 to incentivize the user 102 to increase his/her participation in the health and wellness program 126. As has been shown, the health engine 114 implements the method for the real-time fitness tracking and scheduling, which includes providing both a macro-analysis and a micro-analysis of health data 108 of the user 102, assessing the health data 108 in the if-then scenarios 120 to determine user-specific wellness actions for the user 102 to complete to achieve user-specific health goals, and generating a health and wellness program 126 based on these user-specific wellness actions to achieve the user-specific health goals.

It should be appreciated that the health engine 114 may track all data related to the health and fitness of the user 102 continuously and for the long-term. For example, the health engine 114 may track the health data 108 of the user 102, the first health goal 110 of the user 102, the second health goal 112 of the user 102, the user's participation towards execution of the first wellness action 122, the user's participation towards execution of the second wellness action 124, and the user's level of participation in the health and wellness program 126. The tracking by the health engine 114 of such data related to the health and fitness of the user 102 may provide consistent and real-time feedback on the progress of the education process (e.g., the creation of user awareness and the generation of self-directed education based on the user-specific health goals), the assimilation process (e.g., the transformation of the education into actionable items, such as the first wellness action 122 and/or the second wellness action 124) for the user 102, and the automation process (e.g., the transformation of the actionable items into habits of the user 20 102 to create a healthier individual).

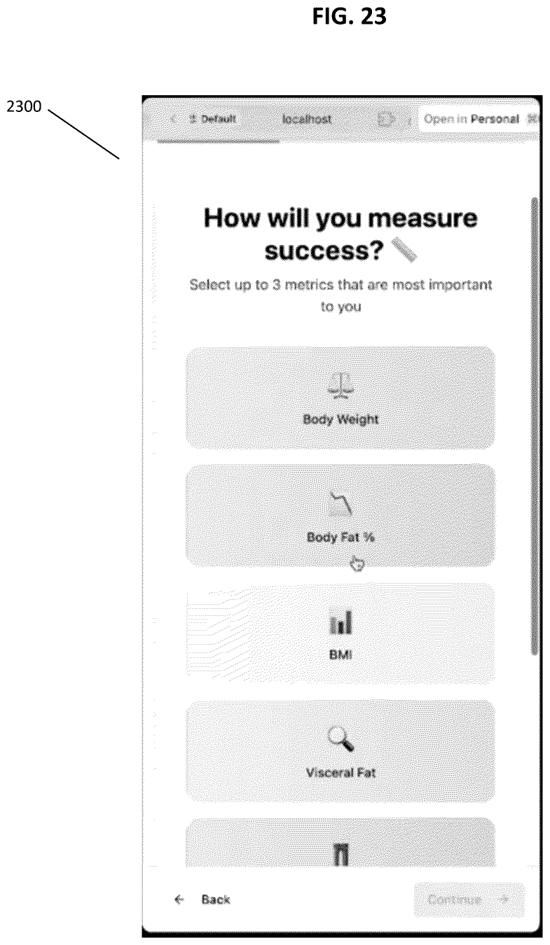

FIG. 23 illustrates an example interface 2300 displayed by a computing device executing an application referred to as Frequent Help, a fitness and health support system configured to assist users and their trainers in managing personalized progress metrics. The graphical user interface (GUI) presents a user onboarding screen in which the user is asked to define how they will measure success. In this screen, the application prompts the user to select up to three metrics that are most important to them. These metrics may be selected from a scrollable list of body composition or health-related indicators, such as body weight, body fat percentage, body mass index (BMI), and visceral fat. The interface is structured to visually separate each metric as a selectable graphical card, each with an associated icon and descriptive label, allowing the user to easily understand and interact with the options presented.

The metric selection interface is an interactive screen within a multi-step onboarding process. In prior screens, the user may have provided motivational input, such as selecting from a list of goals (e.g., reduce weight, increase strength, improve endurance), or described their current activity level, health concerns, or training preferences. In the screen, the selection of progress metrics serves as an input to configure subsequent stages of data collection, analysis, and visualization. These selected metrics are used to populate and prioritize the user's health profile and influence how the system allocates resources for tracking and feedback.

In some embodiments, the selectable metrics may overlap in underlying physiological inputs or calculated parameters. For example, a user may choose both body fat percentage and visceral fat as success indicators, even though both rely on impedance measurements and body composition algorithms. Similarly, body weight and BMI may be selected together, despite both relying on body weight as a shared input. This overlapping is intentional, as the system supports flexible user-defined prioritization and accommodates cases where users wish to track multiple dimensions of the same or related physiological domains. The system allows the user to define what success looks like from their own perspective, and then uses this configuration to drive visualization, alerts, and data capture strategies.

The GUI may operate in conjunction with a wearable device or connected health sensors that collect physiological or biochemical measurements such as heart rate, resting metabolic rate (RMR), oxygen consumption (VO2), body fat, visceral fat, muscle mass, body water, BMI, bone mass, and blood glucose level. These measurements may be gathered through one or more sensor modalities, including impedance sensors, weight scales, photoplethysmography (PPG) sensors, accelerometers, or continuous glucose monitors. Upon metric selection, the GUI communicates with the underlying health engine to adjust the sampling frequency, storage parameters, or display layers associated with each selected metric. This coordination ensures that relevant biometric data are captured and displayed in alignment with the user's selected definitions of progress.

In one example, a user may select body weight, visceral fat, and BMI. The system may then prioritize periodic synchronization with a smart scale and impedance meter, tag and store each metric in time-series format, and generate visual progress charts weighted toward the selected indicators. If the user later changes their selected success metrics to, for instance, muscle mass and body water, the system reconfigures to emphasize different sensors and visual templates, without requiring the user to restart or discard past data. The user interface represents a technical implementation of an adaptive onboarding sequence in which user selection directly informs both downstream data processing and visual representation. Rather than relying on a fixed dashboard or universal tracking scheme, the interface provides a mechanism for user-driven configuration that modifies how data is interpreted, visualized, and prioritized within the application. In some embodiments, the GUI may also be adapted to the user's learning style or accessibility preferences, for example by displaying graphs, numeric targets, or animated illustrations to represent the selected success metrics.

In summary, FIG. 23 illustrates a system-integrated user onboarding interface in which user preferences for progress tracking are not merely recorded, but directly utilized to configure health data collection, interface behavior, and system responses. The use of selectable metrics such as body fat percentage, visceral fat, and BMI provides a tailored user experience aligned with individual health goals, while the modular GUI architecture supports extensibility to additional measurement categories as new sensors or priorities are introduced.

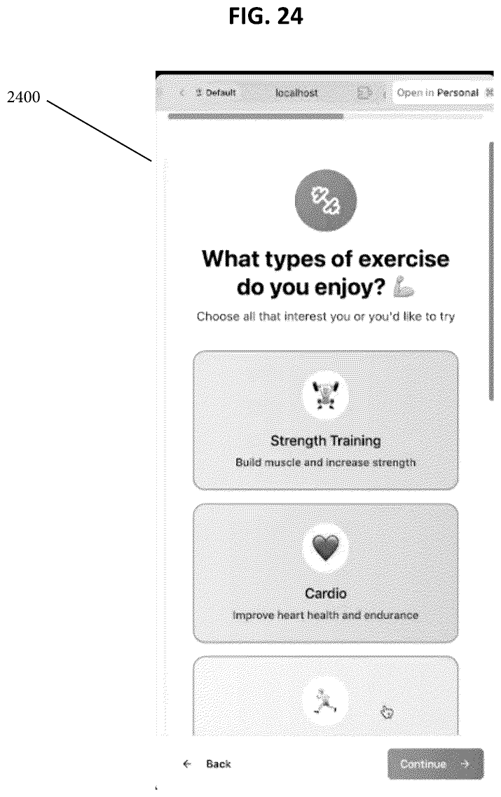

FIG. 24 illustrates a graphical user interface (GUI) 2400 rendered on a computing device, as part of the onboarding flow of a fitness application. In this step, the GUI prompts the user with a question, “What types of exercise do you enjoy?” and displays a set of selectable tiles, each representing a distinct category of exercise. These tiles are presented visually with icons and supporting subtitles to assist the user in understanding the nature of each category. For example, the “Strength Training” tile includes the descriptor “Build muscle and increase strength,” while “Cardio” is labeled “Improve heart health and endurance.” Users are invited to select one or more of these options to indicate interest or willingness to try various exercise types.

In some embodiments, the interface is not limited to mutually exclusive selections; rather, it is designed to recognize and leverage overlapping exercise categories. For instance, a user may select both “Cardio” and “HIIT,” which share physiological demands such as sustained heart rate elevation and aerobic conditioning. The system interprets this overlap to infer a preference for high-energy workouts and may generate interval-based cardio routines or aerobic circuits that satisfy both selections. Similarly, if a user selects “Strength Training” and “Bodyweight Circuits,” the system may recognize the shared emphasis on muscular endurance and hypertrophy, adapting recommendations to include progressive calisthenics or metabolic resistance training.

The system is also capable of interpreting complementary but diverse selections. For example, a user may choose both “Yoga” and “Powerlifting,” which target flexibility and maximum strength, respectively. In such cases, the application may propose an alternating schedule-perhaps combining mobility-focused recovery days with strength-intensive training sessions—to create a holistic program. Likewise, if a user indicates interest in “Pilates,” “Mobility,” and “Stretching,” the application may infer a goal of improving core stability and joint range of motion and recommend a sequence of low-impact, flexibility-enhancing routines.

The GUI supports classification across multiple dimensions, such as type, goal, and intensity. For example, “Cardio” may be associated with endurance, weight loss, and cognitive clarity, and this tagging allows the system to tailor recommendations based on a user's stated objectives. A user who selects “Bodyweight Training,” “Calisthenics,” and “Strength Training” may be presented with programs that emphasize equipment-free strength development, including structured progressions involving push-ups, pull-ups, and isometric holds. If a user chooses “Athletic Conditioning,” “Endurance,” and “HIIT,” the application may respond with a sport-specific regimen that incorporates agility drills, tempo runs, and VO2 max intervals.

Overall, FIG. 24 depicts a GUI configuration that captures nuanced user preferences and translates them into multidimensional fitness profiles. These profiles can inform the generation of personalized programs that combine overlapping modalities in novel ways. The interface enables the collection of subjective interest data that, when combined with performance metrics or biometric feedback, can guide real-time adjustments to the user's regimen. This onboarding interaction thus serves not only as a preference capture mechanism but also as a foundational component of the application's adaptive and goal-aware training logic.

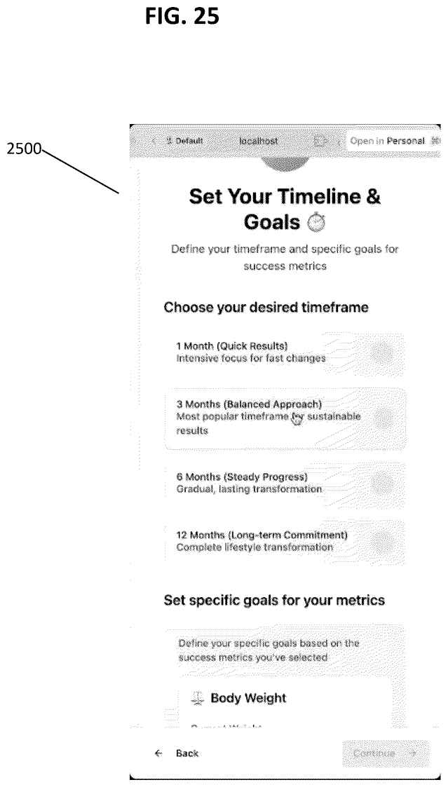

Referring to FIG. 25, a device 2500 with a display presents a graphical user interface (GUI) that facilitates an interactive goal-setting sequence of the AI Trainer Application. In this interface, the user is prompted to define a target timeframe and associate that timeframe with one or more personalized success metrics. These metrics may have been selected in previous steps of the onboarding sequence, such as body weight, body fat percentage, muscle mass, or activity-based goals like weekly workout frequency.

The timeframe selection interface includes a plurality of selectable options, each corresponding to a different duration and descriptive goal path. Example durations include: “1 Month (Quick Results),” “3 Months (Balanced Approach),” “6 Months (Steady Progress),” and “12 Months (Long-term Commitment).” In some embodiments, these options are not mutually exclusive; instead, the system enables users to configure overlapping or sequential goals. For instance, a user may select both a 3-month and a 6-month timeframe, where the initial 3-month goal may serve as a checkpoint for foundational changes such as body weight reduction or improved cardiovascular endurance, while the 6-month goal may emphasize gains in muscle mass or habit reinforcement.

Such overlapping goal configurations allow for dynamic transformation strategies that evolve with user progress. For example, a user targeting a reduction of 10 pounds of body weight over a 3-month period may also designate an extended goal of 20 pounds over 6 months. In another example, a user aiming to reduce their body fat percentage from 25% to 22% within 1 month may concurrently target a long-term goal of reaching 18% over 6 months and 15% over 12 months. The application may track these overlapping time-based objectives in parallel, adjusting recommendations, reminders, and visual feedback accordingly.

The overlapping goals may be further stratified by metric. For example, a user may aim to reach a body fat goal within 3 months, increase muscle mass over 6 months, and maintain overall body weight throughout a 12-month transformation period. Similarly, a user may define shorter-term goals related to habit formation, such as completing 12 workouts in the first month, while also establishing a 6-month goal of consistent exercise adherence (e.g., exercising at least 3 times per week for 24 consecutive weeks). In this way, the AI Trainer Application enables multi-tiered progress tracking that supports short-, mid-, and long-term objectives concurrently.

Upon goal entry, the system stores the user's selections and integrates them into a profile accessible through the user portal. The portal may display visualizations of goal progression, including segmented timelines, milestone markers, and completion percentages, thereby reinforcing motivation and allowing the user to visualize success across multiple scales of time and multiple metrics simultaneously. These timeframes and tiered goal structures support a highly customizable user experience that adapts to diverse fitness objectives, from quick aesthetic changes to deeper, long-term health transformations. The GUI's flexibility in configuring and visualizing such overlapping goals promotes adherence and provides the technical foundation for machine-learning-driven recommendations, which may evolve based on how users perform against short- and long-term benchmarks.

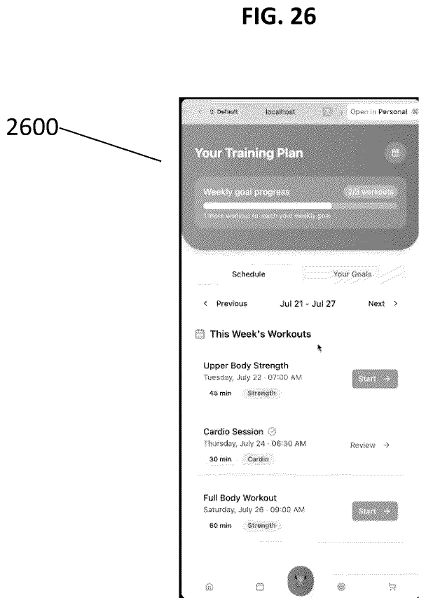

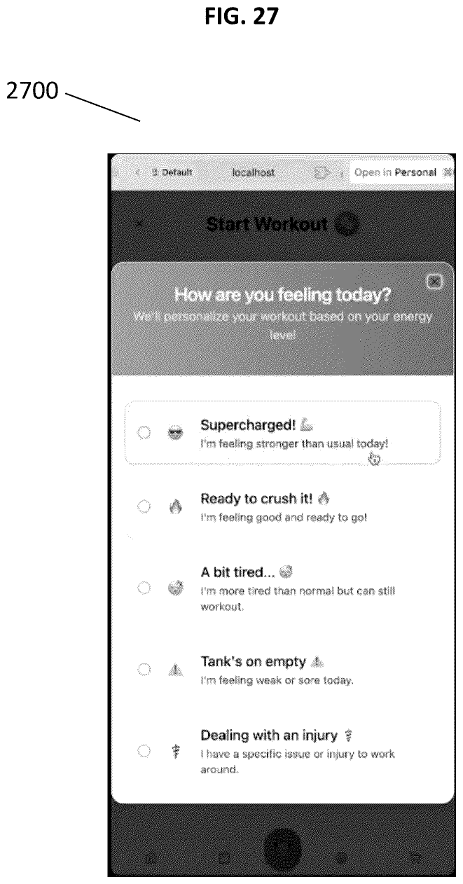

Referring to FIG. 26, a computing device 2600 is depicted with a display presenting a graphical user interface (GUI) corresponding to a personalized training plan view within an AI Trainer Application. The GUI is rendered as part of a mobile or web-based platform and is configured to display dynamic content and update user progress in accordance with stored workout schedules and fitness-related goals. The interface includes both temporal and categorical organizational features to facilitate efficient user interaction. In one embodiment, the training plan is displayed with a section labeled “Weekly goal progress,” which visually represents a comparison between a user's target number of weekly workouts and the actual number of completed workouts. This progress bar may update automatically based on recorded sessions or manual user input. For example, a user may set a goal to complete four workouts per week, and the system may display progress as “3/4 workouts” upon completion of three sessions, enabling near real-time adherence tracking.

The interface further includes a segmented list of workouts scheduled for a particular week, for instance, between July 21 and July 27. Each workout entry includes a heading with the workout type (e.g., “Upper Body Strength”), a scheduled time (e.g., “Tuesday, July 22-07:00 AM”), a session duration (e.g., “46 min”), and a categorical tag (e.g., “Strength”). Action buttons allow the user to either “Start” or “Review” a session. This facilitates both proactive session initiation and retrospective performance review. Examples of scheduled sessions may span overlapping duration ranges. For instance, strength training sessions may range from 40 to 75 minutes, with some intensive full-body workouts scheduled for 60-75 minutes, and shorter targeted sessions (e.g., upper body or core) lasting between 35-55 minutes. Cardio sessions may range from 20-45 minutes depending on the desired heart rate zone, with interval-based runs scheduled for 30-40 minutes and steady-state sessions for 25-50 minutes.

The system may present overlapping ranges in the context of dual-mode workouts as well. For example, a hybrid workout that combines cardio and strength elements may last 50-60 minutes, falling within the upper range of typical cardio sessions and the lower range of extended resistance routines. Similarly, the platform may include mobility or recovery sessions scheduled between 15-30 minutes, which can overlap with lower-end cardio ranges. The application may further include support for alternating-day plans where users are shown workouts with cumulative durations that vary based on weekly goals. A user aiming for three 60-minute sessions and two 30-minute sessions per week may be shown a plan balancing heavy and light effort days. The training engine dynamically adjusts the scheduled activities and durations based on user preferences, recovery data, historical compliance, or recommendations from a trainer dashboard.

In addition to the “Schedule” view, the GUI allows toggling to a “Your Goals” tab where users may input, modify, or track quantitative objectives (e.g., increasing weekly strength volume by 15%, improving VO2 max, or achieving consistency over 6 weeks). The updated goals may recalibrate the training plan's frequency, intensity, and duration metrics to maintain alignment with personalized fitness trajectories. Together, the interface elements represent a feedback-driven training management system designed to visually guide and adapt user progress using dynamic GUI components, schedule forecasting, and contextual data overlays. The approach integrates personalized scheduling, adaptive goal management, and real-time visual progress indicators, all of which may be rendered and managed within a single unified interface on a mobile or desktop computing device.

FIG. 27 illustrates a screen 2700 of a mobile or tablet computing device displaying a graphical user interface (GUI) for a user-interactive module of an AI Trainer Application. In this embodiment, the screen prompts the user to self-report their perceived energy level before beginning a workout. The application dynamically adjusts the workout session based on the selected input, which personalizes the workout intensity, duration, and modality to accommodate the user's readiness level and overall wellness. The energy level selection interface may include discrete inputs along a spectrum of physical and mental readiness. For example, the user may select from “Supercharged!” indicating exceptional readiness, “Ready to crush it!” indicating above-average motivation and energy, “A bit tired . . . ” indicating mild fatigue but willingness to proceed, “Tank's on empty” indicating low energy or soreness, and “Dealing with an injury” indicating pain or physical limitation. These categories allow the user to communicate nuanced physiological or psychological states to the AI Trainer Application.

In certain embodiments, the system uses overlapping intensity thresholds to fine-tune adaptive training decisions. For instance, a user selecting “Supercharged!” may trigger the algorithm to increase planned workload intensity to 90-100% of the user's baseline training maximums, while “Ready to crush it!” may activate a moderately high workload of 75-90%. However, if the user's historical performance indicates high adaptability, even a “Ready to crush it!” input may prompt the system to propose 80-95% intensities. Likewise, “A bit tired . . . ” may lead to reductions in load or volume, suggesting workouts at 50-70% effort or substituting compound movements with simpler alternatives. For example, barbell squats may be replaced with goblet squats or bodyweight variations. “Tank's on empty” may trigger an automatic switch to a 20-40% effort plan with optional recovery modalities such as yoga, foam rolling, or guided stretching sessions.

The ranges may be configured to overlap and blend, using fuzzy logic or tiered rule-based systems. For example, a 60% perceived exertion threshold may be shared between “A bit tired . . . ” and “Tank's on empty,” allowing the system to use contextual data such as sleep score, resting heart rate, and hydration level to finalize the selection. Additionally, a user who selects “Dealing with an injury” may be rerouted to a specialized injury-friendly track, with recommendations that entirely remove stress from the affected area. For example, a user with a shoulder injury may be directed away from pushups and overhead presses toward lower-body and core-focused workouts. In further embodiments, the application may receive biometric inputs from wearable sensors or third-party health data integrations. These inputs may include objective measurements such as heart rate variability (HRV), sleep efficiency, or blood glucose variability, which are then cross-referenced with the subjective energy input. When such data conflict (e.g., user selects “Supercharged!” but HRV is low), the system may display an advisory warning or offer the user a choice to override the system's recommendation.

Moreover, the system may implement time-based weighting of the selected energy level across weeks or months. For example, repeated selections of “A bit tired . . . ” across 3-5 consecutive days may cause the AI Trainer Application to recommend a deload week, with decreased intensity (e.g., 40-60% of prior loads) and a focus on recovery activities. Alternatively, alternating high and low energy inputs may enable the system to prescribe an undulating periodization model, where intensity fluctuates in a planned fashion to avoid overtraining while preserving performance progress. These layered and overlapping energy-state inputs, when processed through an adaptive training engine, enable personalized and context-sensitive training sessions that go beyond static or rule-based fitness routines. Such techniques offer technical improvements in dynamic content generation, real-time decision-making, and individualized coaching logic based on physiological and psychological context, implemented through computing systems operating on user devices.

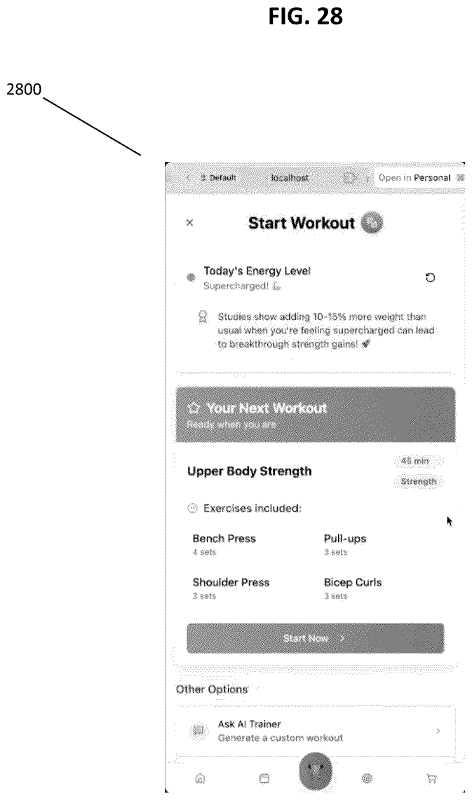

Referring now to FIG. 28, the figure illustrates an example screen 2800 of a computing device operating an AI Trainer Application, configured to assist a user in executing a personalized fitness program. The GUI in FIG. 28 displays a “Start Workout” interface, where a user may review key contextual parameters for the workout session about to begin. These parameters may include the user's reported energy level, relevant scientific insights, and details of the customized exercise set for the day. In some embodiments, the AI Trainer Application adapts workout intensity based on an energy input received from the user, as shown in FIG. 27, and displays tailored recommendations on screen 2800. For example, if the user reports feeling “Supercharged,” the GUI may show a motivational insight, such as “Studies show adding 10-15% more weight than usual when you're feeling supercharged can lead to breakthrough strength gains.” Such recommendations may also vary dynamically based on performance history, rest patterns, and biometric data.

Beneath the energy indicator, the screen displays a training plan module titled “Your Next Workout,” which includes a breakdown of a targeted workout routine, such as an “Upper Body Strength” session. The GUI displays duration (e.g., 46 minutes), the workout type (e.g., Strength), and a list of selected exercises with corresponding set counts. In the illustrated example, the included exercises are “Bench Press-4 sets,” “Shoulder Press-3 sets,” “Pull-ups-3 sets,” and “Bicep Curls-3 sets.” Additional or alternative exercises may include overhead presses, incline bench presses, dips, upright rows, or lat pulldowns, and may be assigned set ranges overlapping or varying by 2-5 sets depending on user experience level, such as beginner (2-3 sets), intermediate (3-5 sets), or advanced (4-6 sets).

The system may also calculate target rep ranges per exercise dynamically, such as 6-8 repetitions for strength-building routines, 10-12 repetitions for hypertrophy, or 15-20 repetitions for endurance, and these may be suggested via the GUI in a popup or expandable module. Ranges may also overlap in hybrid programs (e.g., 8-12 reps) to account for mixed goals such as fat loss and muscle retention. Rest periods may be visually embedded within the UI and dynamically assigned based on the user's energy level and recovery tracking (e.g., 30-60 seconds for hypertrophy, 90-180 seconds for strength, or adjusted based on heart rate recovery data).

In some implementations, additional interface options such as “Ask AI Trainer” may be provided at the bottom of the GUI. This option may enable the user to generate a customized workout using AI-driven recommendations that incorporate ongoing feedback from user inputs, wearable sensors, prior performance trends, recovery states, and training history. FIG. 28 illustrates how high-level workout planning, user state personalization, and detailed, structured data presentation converge to support an interactive and adaptive fitness experience powered by the AI Trainer Application. The GUI enables users to not only view static plans but also interactively refine and execute them in real time.

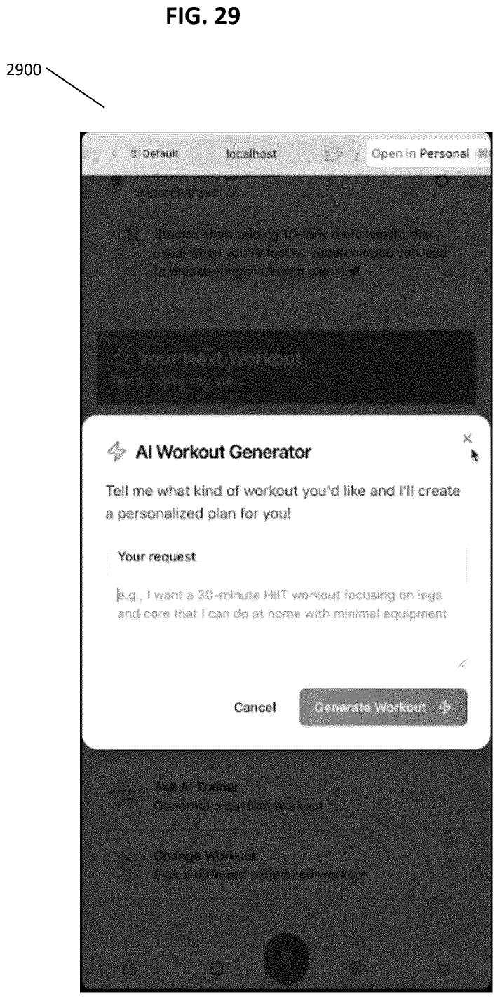

Referring now to FIG. 29, a computing device 2900 is shown with a display rendering a graphical user interface (GUI) component labeled “AI Workout Generator.” This GUI is configured to receive freeform, natural language input from a user and initiate generation of a personalized workout plan. In some embodiments, the GUI allows a user to enter textual requests such as “I want a 30-minute HIIT workout focusing on legs and core that I can do at home with minimal equipment.” This user input is captured and processed by one or more application components executing on the device or on a remote server in communication with the application via a network interface.

The AI Trainer Application includes a natural language processing (NLP) module that parses unstructured textual input to extract structured workout parameters. These may include, for example, duration (e.g., 20-40 minutes), target body region (e.g., legs, core, full body), training modality (e.g., strength, cardio, high-intensity interval training), equipment availability (e.g., bodyweight-only, resistance bands, dumbbells), and location constraints (e.g., home, gym, outdoors). The parsed parameters are used to query an indexed exercise library or structured database storing metadata associated with multiple exercise routines.

The application employs a plan generation engine to construct a customized workout plan based on the identified parameters. For example, if the user specifies a 30-minute HIIT session targeting legs and core with minimal equipment, the engine may retrieve and assemble exercises such as bodyweight squats (4-6 minutes), plank holds (3-5 minutes), glute bridges (3-6 minutes), high-knees (2-4 minutes), lunges (4-6 minutes), and mountain climbers (3-5 minutes). These exercises may be selected to collectively fall within a range of 26-34 minutes, ensuring variability and intensity while remaining within the requested duration window.

In some implementations, the plan generation engine may include a prioritization algorithm that scores exercises based on user-specific attributes including training history, prior performance metrics, fatigue levels, and user preferences. This scoring system may weight factors such as novelty (to avoid repetitive routines), perceived effort (from historical feedback), and rest periods, resulting in a tailored exercise sequence that reflects both immediate user input and long-term adaptive tuning. The selected exercises are further organized into a progression sequence that may balance intensity, duration, and muscle group recovery.

The personalized workout plan is rendered back to the GUI for user review, and includes exercise titles, repetition or time guidance, required equipment, and estimated completion time. The user is presented with options to accept the workout, modify input criteria, or regenerate the workout with different constraints. Upon selection of the “Generate Workout” button, the backend modules execute the parsing, scoring, and plan generation steps described above in real time, enabling a responsive, user-centered customization process.

The AI Trainer Application is architected to perform multi-step input processing and dynamic workout assembly on a per-user basis. The system performs operations that go beyond static content retrieval by incorporating context-sensitive data handling, adaptive rule-based selection, and decision-making processes tied to user interaction, thereby enhancing the overall functionality of the application. The end result is an interactive and personalized fitness training workflow executed through software modules that coordinate across input processing, data querying, logic execution, and GUI rendering.

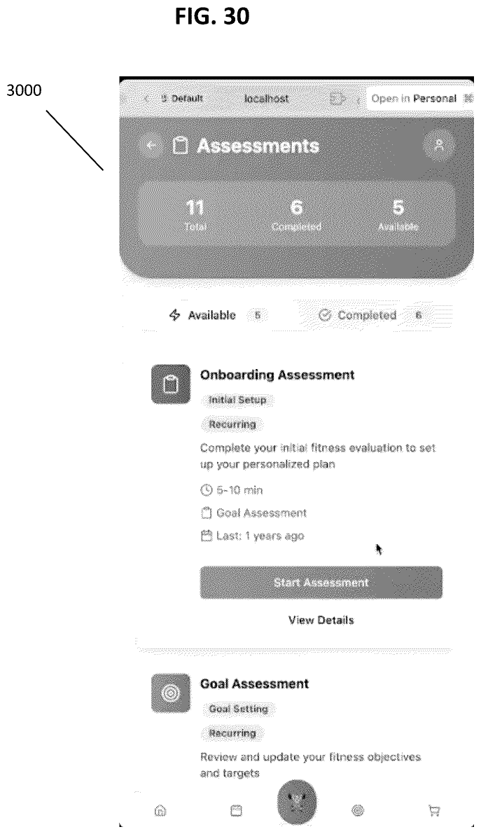

FIG. 30 illustrates a portion of an AI Trainer Application executing on a computing device 3000, where the display presents an “Assessments” interface as part of the user experience. The interface provides structured access to a range of assessment types used to personalize and update a user's fitness program. Graphical elements are configured to display the number of total assessments, completed assessments, and available assessments, each dynamically updated in response to user interactions and backend analytics. The GUI includes assessment modules such as an onboarding assessment and a goal assessment, both of which may be recurring and time-sensitive.

The onboarding assessment enables users to complete an initial evaluation that informs generation of a personalized training plan. This process may involve questionnaire inputs, biometric measurements, or movement assessments, and typically takes between 5 to 10 minutes. The goal assessment prompts users to reflect on and revise fitness objectives based on progress tracking data. The system retains timestamps, status flags, and goal metadata associated with each assessment session, thereby allowing longitudinal monitoring and dynamic plan refinement.

In some embodiments, additional assessment modules may be included to evaluate specific exercise techniques and movement forms. These modules may utilize device-integrated sensors, such as accelerometers or cameras, to capture and analyze user motion in real time, enabling feedback on posture, form accuracy, and risk of injury. The system may provide corrective recommendations, flag deviations from safe biomechanical patterns, or adjust training plans accordingly. The AI trainer processes assessment inputs using a combination of deterministic logic and machine learning models. Assessment results are stored in structured data objects, which are parsed and ranked based on scoring rubrics and model-driven insight generation. These data objects are then used to update user-specific fitness plan parameters, such as intensity, duration, exercise selection, and rest intervals. The AI trainer continuously reprocesses this assessment data in conjunction with historical performance trends, user preferences, and program engagement metrics to generate evolving recommendations.

To further support users in managing their fitness journey, the application provides access to an integrated Explore Hub. The Explore Hub acts as a centralized resource repository, allowing users to browse between different workout templates, educational lessons, recommended products, and assessments. The design of the Explore Hub is intended to streamline the fitness experience by minimizing decision fatigue and enabling the user to efficiently allocate time toward activities that yield the highest positive impact. The system may rank or filter available resources based on user context, such as past activity, goals, and recent assessment performance. The assessments interface and Explore Hub form a closed-loop system that combines user self-reporting, sensor-based observation, dynamic feedback, and intelligent resource delivery to enhance engagement, safety, and goal attainment in personalized fitness programming.

In some embodiments, the disclosed system implements an AI Trainer platform configured to generate, update, and optimize individualized fitness programs based on user-specific inputs, assessments, and real-time biometric or behavioral feedback. The platform comprises a computing system having a graphical user interface (GUI), a rule-based and machine learning-driven backend, and various data acquisition modules configured to receive both structured and unstructured input from a user or associated sensing devices. The AI Trainer includes a virtual assistant component, herein referred to as the “AskAI Chatbot,” which serves as a conversational interface through which users can request modifications to their training schedule, inquire about fitness principles, or initiate personalized program generation. The chatbot may respond to direct textual queries, detect deviations from ideal form via video input, and trigger system-level updates to a user's program based on detected changes in subjective or objective status.

### Chronic and Acute Program Generation

The AI Trainer system supports both chronic (e.g., 8-12 week periodized training programs) and acute (real-time or near-real-time adjustments) workflows. In one embodiment, the chronic training protocol is initiated based on client Intake and Assessment data. Intake data includes self-reported information such as fitness goals, medical limitations, and preferred equipment, while assessment data may be obtained through manual inputs or video recordings of physical assessments, such as squats, planks, hip bridges, and push-ups. These video assessments are analyzed by a form detection engine that applies spatial tracking algorithms and computer vision to produce a form deviation heatmap. The heatmap identifies biomechanical inefficiencies, such as premature anterior knee translation during a squat, which are interpreted as indicators of muscular imbalances, such as weak hamstrings and overactive quadriceps.

In some embodiments, the system includes a form detection engine configured to analyze video data of a user performing a prescribed exercise and to generate a form deviation heatmap representing regions of the body that deviate from an ideal biomechanical form. The form detection engine operates as part of a computer-vision pipeline that continuously transforms raw image data into structured biomechanical representations. The engine first receives streaming video input from one or more cameras of a client device, such as a smartphone, smart mirror, or wearable camera. Each incoming video frame is processed through an image normalization module that stabilizes exposure, compensates for variable lighting conditions, and isolates the subject's silhouette from the background using background-subtraction and segmentation algorithms. The normalized frames are then processed by a pose-estimation module that detects skeletal key points, such as hip, knee, ankle, shoulder, and wrist joints, by applying convolutional neural network filters and feature-extraction layers trained on labeled human-movement datasets.

The form detection engine maps the extracted key points into a three-dimensional coordinate space, generating a continuous skeletal model that tracks positional vectors and angular relationships between connected joints over time. From this motion data, the engine computes joint angles, angular velocities, and body-segment trajectories to form a digital motion profile of the user's exercise performance. The computed motion profile is compared against a reference biomechanical template retrieved from a database of idealized movement patterns corresponding to the selected exercise type. Each template includes joint-angle ranges, torque distribution patterns, and time-domain motion curves that define the expected kinematic behavior for that exercise. The comparison produces a deviation vector that quantifies both the magnitude and spatial direction of each detected discrepancy.

The deviation vector is transmitted to a biomechanical inference module that maps the detected deviations to likely muscle-activation imbalances using an anatomical correlation model. This model links specific joint-motion irregularities, such as anterior knee translation or asymmetric hip rotation, to overactive or underactive muscle groups based on empirically derived relationships between kinematic movement and neuromuscular response. The inference module computes activation coefficients for major muscle groups and generates a corresponding activation map that specifies the relative engagement level of each group during the exercise. The coefficients are encoded as numerical values, which are then converted into a visual overlay using a rendering subsystem.

The rendering subsystem generates a form deviation heatmap that is displayed within a graphical user interface. The heatmap includes a human-body outline or three-dimensional avatar and applies a color gradient to depict the magnitude of deviation or imbalance across muscle regions. For example, regions associated with excessive or compensatory activation may be displayed in red or orange, while regions associated with inhibited or under-recruited muscles may appear in blue or gray. The form detection engine continuously updates the heatmap in near real time by streaming recalculated activation coefficients to the rendering subsystem, enabling dynamic visualization of movement corrections as the user adjusts posture or technique. The update latency may be maintained within a range of approximately 100 to 300 milliseconds to ensure perceptible synchronization between physical motion and displayed feedback.

In some embodiments, the system integrates auxiliary sensor data from wearable devices containing accelerometers, gyroscopes, or surface electromyography sensors. The sensor data is time-aligned with the video-derived kinematic data through a sensor-fusion module that applies synchronization algorithms and weighted averaging to improve accuracy in determining actual muscle activation levels. The fused data enhances the reliability of the heatmap representation, allowing the form detection engine to differentiate between visually apparent misalignments caused by camera angle and true biomechanical deviations.

By executing these operations, the form detection engine provides a computer-implemented improvement in motion analysis and user feedback systems. The described architecture achieves reduced processing latency through optimized frame-batch processing, improves precision through multimodal data fusion, and enhances reliability by automatically adapting biomechanical templates based on user-specific attributes obtained from intake data. The combination of these elements produces an adaptive, data-driven heatmap visualization that allows users to receive immediate, objective, and personalized feedback on physical performance within a digital training environment. In some embodiments, the form detection engine applies spatial tracking algorithms and convolutional neural networks (CNNs) to video data of a user performing a prescribed exercise. The system generates a form deviation heatmap, which represents body regions deviating from the ideal biomechanical standard for the given movement. For example, in the case of a bodyweight squat, the CNN may detect an anterior shift of the knees at frame sequences between 5-12 and again between 28-35 of the video sequence. Such premature anterior knee translation is interpreted as a compensatory pattern that may indicate quadriceps overactivation and weak distal hamstrings or gluteal under activation.

In some embodiments, the system includes a form detection engine configured to process video data of a user performing a prescribed physical exercise. This engine performs multi-stage analysis of the video to derive exercise-specific form deviation metrics. Initially, the video input is parsed into individual frames, and a spatial tracking module identifies and tracks a set of skeletal landmarks across the temporal sequence. This module may employ key point detection techniques that produce coordinate vectors corresponding to major joints such as knees, hips, shoulders, elbows, and ankles. These skeletal representations are normalized and used as input to a convolutional neural network (CNN) that has been trained to classify biomechanical form deviations based on labeled datasets of correct and incorrect movement patterns.

The CNN extracts spatial and temporal features from the joint trajectory data, including relative limb angles, velocity vectors, and deviations from standard pose transitions for a given exercise type. These features are processed through convolutional layers, batch normalization, and activation functions such as ReLU to produce a classification output that indicates one or more specific form errors. The CNN may also include recurrent or attention-based modules to improve sensitivity to time-dependent motion patterns, enhancing the system's ability to distinguish between minor and major deviations.

Once the CNN outputs the deviation data, a form deviation heatmap is generated. This heatmap graphically overlays the human body diagram with visual indicators, such as color-coded regions, that reflect the degree of deviation. For instance, regions where the detected form diverges significantly from biomechanical norms may be rendered in red, while less critical areas may appear in blue or green. The heatmap is dynamically updated frame-by-frame as the user continues to perform the exercise, reflecting real-time adjustments based on ongoing pose estimation and CNN inference.

This process is tightly integrated with a health engine that stores normative movement templates for various exercises, user-specific biomechanical baselines (derived from intake questionnaires and past assessments), and programmatic rules for exercise progression or regression. The health engine uses the outputs of the CNN and spatial tracking system to not only generate visual feedback but also to suggest corrective interventions, such as modified exercise prescriptions, cueing prompts, or additional assessments to triangulate suspected muscular imbalances.

By combining spatial tracking and CNN-based analysis in a pipeline that provides structured, real-time feedback derived from user-captured video, the system achieves several technical improvements. These include improved latency in generating personalized exercise feedback, increased precision in identifying complex biomechanical deviations, and enhanced reliability in adapting exercise protocols to individual users. Moreover, because the system relies on real-time visual data combined with adaptive inference models rather than manual observation or abstract goal-setting, it provides a technical solution that is not purely mental or mathematical in nature, and is rooted in concrete data processing steps implemented via a non-generic computing architecture.

The heatmap component dynamically represents these deviations using intensity ranges, where specific muscle regions are encoded using color gradients. For instance, a color gradient ranging from light red to dark red may indicate increasing overuse or excessive load on a particular muscle group (e.g., quadriceps), while light blue to dark blue indicates increasing underuse or inadequate recruitment (e.g., hamstrings or gluteus maximus). In some implementations, additional overlapping zones may be shown in purple, magenta, or orange to indicate simultaneous activation conflicts or asymmetrical force vectors, such as lateral hip deviation combined with excessive spinal extension. In one example, the user's right hip may display a magenta zone (range overlap: red and blue), indicating both abnormal overcompensation (e.g., right quadratus lumborum) and underutilization of stabilizers (e.g., gluteus medius) at the same time during the mid-squat phase.

The system further supports multiple heatmap thresholds that are conditionally applied based on user data received via intake questionnaire responses and biometric wearables. For instance, if a user reports a history of knee instability and simultaneously presents an RPE rating below 5 after completing three consecutive sets of squats at 70% of 1RM, the heatmap module may widen its deviation sensitivity range from ±10° joint angle variance to ±5° for hip and knee tracking. This ensures more granular detection of misalignment under conditions where joint precision is clinically relevant.

Additionally, the form detection engine may apply overlapping temporal confidence ranges when determining deviation persistence. For example, a transient deviation occurring for fewer than 3 frames out of any 10 may be shown in yellow to indicate non-critical form drift, whereas persistent deviations across 7 or more frames within the same interval may be shown in red, regardless of muscle type, signaling high correction priority. One or more classifications may also be used for joint torque anomalies and motion path divergence. For instance, a forward-shifted center of pressure during a deadlift may be mapped as a deviation in both sagittal and frontal planes, and highlighted in a hashed or cross-faded zone across the lower leg and lumbar regions. These ranges provide a more comprehensive visualization that reflects the complexity of real-time biomechanics, enabling the AI Trainer application to recommend precision corrective exercises. The heatmap is not static, it dynamically updates during playback or live performance and can adapt based on new sensor input or subjective user feedback collected post-exercise.

The disclosed system provides several technical benefits that improve the operation of computer-based fitness training platforms. First, the integration of a form detection engine with real-time video analysis enables enhanced biomechanical feedback by continuously updating a form deviation heatmap. This heatmap visually identifies regions of the user's body that deviate from an ideal biomechanical pattern using computer vision and spatial analysis, enabling corrective action in real-time rather than post-session review. Such continuous visual feedback represents an improvement in system latency and precision, as the feedback is generated dynamically and delivered to the user without delay, allowing form corrections to be implemented during the exercise.

Moreover, the system employs multimodal sensor fusion, drawing from both video data and biometric sensor inputs, such as accelerometer data from a wearable device. This data is processed alongside structured intake data and prior assessments to generate personalized biomechanical insights. By combining subjective and objective data streams, the system produces adaptive feedback that is more precise and reliable than generic fitness applications. Notably, the heatmap classification is not binary; instead, it includes overlapping ranges of muscular activation, imbalance detection, and form deviation severity, which together allow for a multivariate classification of movement deficiencies.

The system's use of convolutional neural networks (CNNs) is not generic but is applied in a specific technical context. The CNNs are trained to identify discrete biomechanical faults, such as anterior pelvic tilt, valgus knee collapse, or spinal flexion, by analyzing frame-by-frame video of user movement. These patterns are compared to biomechanical norms and stored templates, and the system translates deviations into a color-coded heatmap for user visualization. This technical implementation allows the CNNs to convert unstructured image data into structured form deviation feedback, thereby improving the efficiency and scalability of personalized training recommendations.

Furthermore, the system includes a closed-loop architecture wherein feedback from the heatmap and sensor data dynamically influences the user's workout plan through a health engine. This closed-loop system ensures the platform remains responsive to both acute and chronic changes in user condition, such as reduced energy, changes in equipment availability, or fatigue. The ability to adapt the workout prescription in real-time based on detected form deviations and user feedback enhances the reliability and robustness of the training program, mitigating injury risk and optimizing performance outcomes.

Finally, the system architecture combines edge computing and cloud-based processing to optimize both performance and energy efficiency. Latency-sensitive operations, such as pose estimation and video frame analysis, are executed locally on the user device, while inference modeling, plan adaptation, and biometric trend analysis are offloaded to a remote health engine hosted in the cloud. This hybrid computing architecture reduces computational strain on the client device while maintaining a seamless user experience. The integrated user interface (GUI), together with the real-time heatmap feedback, form detection engine, and health engine, represents a practical and technical improvement in the field of digital fitness systems. These improvements are rooted in specific system architecture and data processing techniques and are not performed in the human mind or through generic computer functionality.

When the user initiates a video assessment, image frames are captured from a front-facing or side-facing camera of a mobile device, wearable device, or connected camera system. Each frame is pre-processed through image normalization routines that stabilize exposure, crop to the subject's silhouette, and detect body orientation. The processed frames are then fed into a pose-estimation model trained to identify skeletal key points (e.g., shoulders, elbows, hips, knees, ankles) in two- or three-dimensional space. The model may be implemented using convolutional neural networks or transformer-based architectures optimized for human-motion tracking.

The engine assembles a continuous skeletal motion trajectory from sequential frame data. Using this trajectory, the system computes joint angles, angular velocities, and segment vectors for major body regions (torso, arms, legs). Each parameter is compared against a stored biomechanical baseline template corresponding to the target exercise. For example, a stored template for a barbell squat includes expected hip-to-knee flexion ratios, spine alignment vectors, and vertical displacement thresholds.

If the engine detects an angular or timing deviation exceeding a threshold—for instance, an anterior knee displacement greater than 5° beyond the toe line or early hip flexion offset—the deviation is recorded and spatially mapped to the affected joint coordinates. The system classifies the deviation type (e.g., forward-knee translation, lumbar rounding, lateral shift) and cross-references it with an anatomical activation map that links deviation patterns to specific muscle imbalances or overcompensations. For example, excessive knee translation may be associated with weak distal hamstrings and overactivation of the quadriceps, while lumbar rounding during a hip hinge may correspond to underactive gluteals and over-reliant spinal erectors.

Using this inference, the form detection engine generates a form deviation heatmap, a graphical overlay displayed on a body-diagram GUI. The heatmap depicts muscle groups with color intensities proportional to detected deviation magnitude or inferred overactivation. Muscles exhibiting excessive or compensatory activation may be displayed in red or orange, while inhibited or under-recruited regions are shown in blue or gray. The user interface allows rotation of the virtual body model to view anterior, posterior, and lateral planes.

The system operates dynamically by updating the heatmap in near-real time as the user continues the movement. Each new frame triggers recalculation of joint angles and deviation magnitudes, which the rendering module uses to refresh the displayed heatmap within a latency window of approximately 100-300 milliseconds. This provides the user with immediate visual feedback on form quality. The health engine may also trigger adaptive corrective guidance, such as textual prompts (“Shift hips backward”), audio cues, or short overlay animations demonstrating proper alignment.

To improve reliability, the system combines video-based pose data with auxiliary sensor inputs from connected wearable devices. For example, inertial measurement units (IMUs) in a smartwatch or thigh-mounted sensor provide acceleration and orientation vectors that confirm movement direction and speed, while heart-rate or surface-EMG data indicate physiological activation intensity. These signals are fused with the video data through a sensor-fusion algorithm that time-aligns the data streams using timestamp synchronization. When fused, this multimodal dataset allows the form detection engine to validate whether detected visual deviations correspond to actual muscular imbalance or simply to camera-angle distortion.

The engine's output is stored as a structured data object containing joint-angle arrays, activation coefficients, deviation classifications, and corrective recommendations. These data are used to refine subsequent exercise sessions. For instance, if repeated assessments show consistent quadriceps dominance during lower-body exercises, the system automatically prescribes additional glute-activation or hamstring-strengthening drills in the user's program. Over time, longitudinal analysis of the deviation heatmaps provides a quantitative record of biomechanical improvement.

This combination of video-based skeletal tracking, sensor-fusion validation, biomechanical deviation mapping, and real-time graphical rendering constitutes a technical architecture that enables precise, automated detection and visualization of form inefficiencies. It provides a concrete implementation pathway for dynamic heatmap generation, specifying how the data are acquired, processed, fused, and rendered by the health engine to assist the user in corrective exercise performance.

When a form anomaly is detected, the AI Trainer may prompt the user to complete additional assessment modules (e.g., squat mobility or dynamic balance tests) to triangulate the observed data. The resulting decision tree may prescribe corrective exercises. For instance, if a user experiences back discomfort during a hip bridge, the system may recommend glute activation exercises such as single-leg bridges, clamshells, or side-lying hip abduction, followed by integrative movements such as a band-resisted bridge.

### Prompt Generation and Auto-Templating

Based on assessment data, a prompt is constructed and processed by the AI Trainer engine to produce a tailored training plan. While prompts may initially be created manually, system logic is configured to dynamically generate prompts using templated intake and assessment formats. This includes both chronic planning and acute response layers. The AI Trainer system stores prompt templates linked to specific user profiles and dynamically updates them upon receipt of new input. As the system collects more data, the prompt generation process becomes increasingly automated and self-adaptive.

In some embodiments, the AI Trainer system includes a prompt generation and auto-templating architecture designed to automate the creation of personalized training plans by combining structured assessment data with dynamically updated logic modules. The process begins with a set of structured intake and assessment forms presented to the user through a graphical user interface (GUI). These forms capture a variety of subjective and objective inputs, such as user-identified training goals, prior injury history, perceived weaknesses, current energy levels, available equipment, and performance on physical assessments. The performance data may include video recordings of the user executing movements (e.g., squats, hip bridges, push-ups), from which spatial and biomechanical information is extracted via a form detection module. This module applies computer vision algorithms and pose estimation models to identify joint angles, alignment, and muscular recruitment deviations. The resulting biomechanical indicators are converted into structured assessment tags and scores that are stored in a user profile database.

The prompt generation module accesses these structured inputs and inserts them into predefined prompt templates stored in a template library. Each template is a modular construct composed of multiple parameterized segments, such as {training goal}, {movement_dysfunction}, {equipment_constraints}, and {mobility_score}, each of which is bound to a corresponding field within the intake/assessment data schema. The system uses logic-based mapping rules and conditional branching to match templates with appropriate user categories. For example, if a user is flagged as “postpartum rehabilitation” with “limited ankle dorsiflexion,” the prompt template selected will differ from one used for a “high-performance athlete” with “anterior pelvic tilt.”

Once the prompt is populated, it is encoded into a structured representation such as a JSON object and transmitted to the AI Trainer engine, which may reside locally or on a cloud-based inference server. The AI Trainer engine executes a multi-step workflow comprising: (1) ingestion of the prompt, (2) extraction of relevant parameters, (3) plan synthesis using a combination of logic rules and AI-driven suggestion engines, and (4) output formatting for GUI display and long-term storage. The training plan is not simply a list of exercises, but includes volume, intensity, rest intervals, progression criteria, and built-in contingencies (e.g., substitutions if the user reports pain or lacks equipment).

As the system receives new data from the user, whether in the form of updated assessments, biometric feedback from a wearable device, or user-reported outcomes, the prompt generation module triggers a dynamic re-evaluation. For instance, if a user completes a workout and reports a low rating of perceived exertion (RPE), and wearable data confirms high recovery metrics, the system may automatically adjust the prompt by incrementing load or suggesting a progression exercise. Similarly, if the system detects under-recruitment of glutes and over-recruitment of quadriceps via the form detection module, the prompt may include corrective suggestions in the form of activation exercises or warm-up protocols.

Over time, the prompt generation becomes increasingly autonomous through a self-adaptive feedback loop. The system tracks past prompt-plan pairs, compares them with user adherence and progress metrics, and ranks templates accordingly. This allows future prompts to be tailored based on not only current data but historical outcomes, improving the relevance and efficacy of training prescriptions. This data-driven automation reduces latency in generating new programs, increases the responsiveness of the system to real-world user behavior, and minimizes the need for human intervention in crafting personalized fitness plans.

This architecture reflects a technological improvement over conventional static fitness applications. Unlike systems that require manual construction of plans based on user input, the disclosed system automates plan generation using structured templates, AI inference, and real-time adaptation to user state. The interaction between the templating engine, AI trainer, and health data interfaces results in an integrated, non-generic computing system that improves user engagement, reduces configuration time, and delivers precision-targeted exercise protocols based on real, measurable inputs. These features are tied to a specific technological implementation that includes defined data flows, structured processing, and adaptive automation that would not be practically achievable by mental processes alone or by conventional pen-and-paper planning.

### Acute Response System Based on Subjective and Objective Metrics

In the acute response layer, the system responds to changes in either subjective feedback (e.g., user self-reported rating of perceived exertion, or RPE) or objective sensor-based input (e.g., heart rate, motion patterns from wearable devices). For example, if a user performs a strength movement at a consistently low RPE across multiple sessions, the system may trigger progression logic to either increase load, add sets, or replace the movement with a more advanced variant. The AI Trainer backend compares the number of successful completions of a movement at a given intensity across a defined window (e.g., within the past 7 days), in combination with reported RPE, to determine whether adaptation is warranted. If an adaptation is needed, the chatbot may notify the user with a suggestion, such as, “Ready to increase intensity for squats? Consider adding 10% more weight or performing an advanced variation.” The system also accounts for pharmacologically induced limitations to biometric response. For example, when a user is on beta blockers that suppress expected heart rate elevation, the RPE may be used as a surrogate measure for workload adaptation. The AI chatbot is capable of interpreting user RPE input and initiating program updates accordingly. For cardiovascular workouts, if wearable device data indicates attenuated heart rate response over repeated bouts, the AI Trainer may prompt the user to either increase the duration or intensity of the exercise. For example, if jogging fails to elevate heart rate beyond a prior threshold, the chatbot may suggest interval running or incline adjustments, supported by back-end logic mapping biometric metrics to exertion equivalencies.

In some embodiments, the AI Trainer system includes an acute response engine that continuously monitors both subjective and objective data streams to make real-time adjustments to a user's prescribed fitness program. The system architecture includes a feedback acquisition module, a metric aggregation layer, a rules-based progression engine, and an AI-driven chatbot interface that communicates actionable adaptations to the user via a graphical user interface (GUI).

In some embodiments, the AI Trainer system incorporates an acute response engine that is implemented as an adaptive, event-driven computing architecture configured to process heterogeneous user data streams in real time. The acute response engine receives both subjective feedback and objective sensor data through a feedback acquisition module that continuously samples multiple input sources. Subjective feedback may include a user's rating of perceived exertion (RPE), qualitative energy reports, or soreness levels entered through an interactive GUI or voice command processed by the chatbot interface. Objective input may be received from connected wearable devices that continuously transmit heart rate, heart rate variability (HRV), accelerometer data, and motion vectors. The system synchronizes these asynchronous data streams using a timestamp alignment process to ensure temporal correlation between user-reported exertion and biometric measurements.

The synchronized data are fed into a metric aggregation layer that computes rolling averages, weighted deviations, and confidence intervals across overlapping time ranges. For instance, a seven-day window may overlap with a three-day micro-cycle window to capture both acute and short-term adaptation patterns. Within these overlapping ranges, if the user's RPE remains between 3.5 and 4.0 across sessions while heart rate variability improves within a 5-10% band of baseline, the system determines that the training stimulus is suboptimal and that progression criteria may be met. Similarly, if motion pattern consistency (derived from accelerometer and gyroscope readings) exceeds 92% correlation across three consecutive sessions within a ten-day period, the progression engine flags readiness for advancement. These overlapping temporal ranges allow the system to distinguish transient fatigue or poor recovery from genuine long-term adaptation, ensuring that real-time changes are not triggered prematurely or too late.

The rules-based progression engine evaluates these aggregated metrics against predefined conditions encoded as parameterized logic statements. Each rule includes tolerance thresholds and variable weighting functions to prevent noise from influencing the decision process. For example, an advancement trigger may require an RPE under 5.0, HRV above 85% of baseline, and a heart rate recovery time under 45 seconds within any overlapping five-day segment that includes at least three valid training sessions. Conversely, if overlapping data windows reveal an RPE above 7.5 and HRV below 75% of baseline for two or more consecutive sessions, the regression logic reduces workload or replaces the current exercise with a lower-intensity variant. These thresholds and window overlaps are configurable and automatically recalibrated through reinforcement data accumulated during continued user engagement.

Once an adaptation condition is met, the AI chatbot interface dynamically generates a contextual response message that is rendered through the GUI. For example, the chatbot may display or vocalize a message such as, “Your last three strength sessions show consistent recovery and low perceived effort. Would you like to increase your squat load by 5% to 10% or add one additional set?” The message options are generated using a contextual suggestion model that references prior adaptation outcomes, stored in a local performance history database. If the user accepts a suggestion, the system logs the new parameters and updates the next program cycle in the training database. These adjustments are reflected immediately in the exercise scheduling interface, ensuring real-time synchronization between AI-driven recommendations and the user's active training plan.

The acute response engine improves computer functionality by implementing an asynchronous event-driven processing model that reduces latency between data acquisition and adaptive feedback. The overlapping range calculations and weighted rule logic allow the system to generate adaptive recommendations that are both context-sensitive and noise-resistant, addressing a technical problem inherent in traditional static or manually updated training systems. The system achieves improved accuracy by continuously recalibrating sensitivity thresholds based on ongoing data inputs, resulting in a computing architecture that dynamically optimizes user interaction and machine responsiveness. This implementation provides a concrete technological improvement in how fitness data are processed, analyzed, and acted upon by a computing device, rather than a mere automation of mental judgment or abstract decision-making.

### Subjective Metric Handling (RPE-Based Logic):

The feedback acquisition module includes an interface where users self-report subjective metrics such as Rating of Perceived Exertion (RPE) on a per-exercise basis. This input is captured through a structured form rendered by the GUI on a mobile device or wearable interface. Once received, RPE values are timestamped and associated with the corresponding exercise ID, session ID, and user ID, and stored in a time-series user performance database. The metric aggregation layer queries this database to determine if an exercise has been performed a threshold number of times (e.g., three times in the past seven days) with an RPE below a predefined threshold (e.g., RPE≤5 out of 10). If these conditions are satisfied, the progression engine triggers a state transition in the user's training program.

This progression logic is encoded in a decision tree or state machine that defines a range of allowable adaptations. For instance, for strength-based movements, the system may automatically increment the prescribed load by a percentage (e.g., +10%), increase the number of repetitions or sets, or swap the current exercise for a higher-difficulty progression (e.g., replacing goblet squats with barbell front squats). The system logs these transitions for traceability and model retraining purposes.

Objective Metric Handling (Wearable and Biometric Data): In parallel with subjective data collection, the system continuously acquires objective physiological metrics through one or more health monitoring wearables, such as smartwatches, chest straps, or biometric patches. These devices are configured to stream real-time biometric signals, including heart rate (HR), heart rate variability (HRV), and motion vector data (e.g., 3-axis accelerometry and gyroscopic data), to the acute response engine. Data acquisition occurs at configurable sampling frequencies ranging from 1 Hz to 10 Hz, depending on the device's capabilities and the demands of the training context. Upon receipt, the incoming biometric signals are time-stamped and temporarily cached in a local or cloud-based buffer before being passed to a metric aggregation layer.

Within this layer, the signals are normalized and preprocessed to reduce measurement noise and ensure comparability across sessions and users. A Kalman filter or similar recursive estimator is applied to the raw heart rate and HRV streams to smooth transient fluctuations and estimate the true physiological state over time. Simultaneously, motion vector data are segmented into activity bouts using movement classification algorithms that detect patterns consistent with specific exercise modalities (e.g., walking, squatting, cycling). For example, repeated vertical oscillations in the accelerometer's Z-axis, when paired with a stable gyroscopic rotation rate, may be classified as a bodyweight squat movement. These classified activity types are tagged and associated with corresponding biometric segments, allowing the system to determine not only the exertion level but also the movement context in which that exertion occurs.

The resulting processed metrics are stored in a time-series database with overlapping analysis windows, for instance, rolling five-day and seven-day periods, used to evaluate readiness and recovery trends. These overlapping temporal windows allow the progression engine to perform fine-grained comparisons across adjacent training cycles and prevent abrupt or inaccurate adjustments. By integrating multiple data channels (subjective and objective) in a unified analysis pipeline and applying advanced signal processing to extract exercise-specific insights, the system provides a concrete technological improvement over conventional fitness tracking tools. These improvements include enhanced precision in program adaptation, reduced latency in generating tailored feedback, and increased reliability in interpreting user status across varied physiological and contextual conditions. The use of specific algorithms (e.g., Kalman filtering) and structured sensor data workflows ensures that the claimed system does not merely automate mental processes but instead implements a technical solution to the real-world challenge of adaptive fitness programming.

The system maintains a baseline profile for each user, calculated using a rolling average of previous exercise sessions. When the current session's biometric profile diverges from the expected values (e.g., failure to reach target heart rate zones during cardio intervals), the progression engine reevaluates the workout parameters. For example, if the user's heart rate plateaus despite increased workload, the system may prompt the user to adjust the modality (e.g., switch from steady-state running to intervals) or increase incline/resistance settings. These prompts are issued through the AI chatbot interface, which generates suggestions based on a rules-based mapping between physiological signals and programmatic adjustments.

The system maintains a dynamic baseline performance profile for each user, which is calculated using a rolling average derived from historical biometric data collected across prior training sessions. This baseline profile includes parameters such as average peak heart rate, heart rate recovery slope, time in zone metrics, and workload volume correlations, such as average heart rate at given resistance settings or running speeds. These metrics are stored in a time series database and regularly updated by the metric aggregation layer, which applies moving average functions and exponential smoothing to accommodate both long term fitness adaptations and short term fluctuations. When a new training session is initiated, the system compares incoming real time biometric data, such as heart rate sampled at 1 Hz from a chest worn ECG grade sensor, against the stored baseline using statistical divergence calculations, such as z scores and percent deviation thresholds.

If the real time data significantly diverges from expected values, such as failure to enter or maintain a target heart rate zone during an interval session, the progression engine flags the deviation and initiates a reevaluation of current workout parameters. For example, if the user's heart rate plateaus below a target zone, such as Zone 3 at 140 to 160 bpm, despite incremental increases in treadmill speed or resistance, the progression engine interprets this as insufficient workload stimulus or a maladaptive physiological response. Based on rules based logic encoded within the exercise adaptation module, the system may generate a decision tree that offers modality changes, such as switch from steady state running to incline based intervals, or specific parameter adjustments, such as increase resistance level by 5 to 10% or duration by 2 to 4 minutes. These decision trees are mapped to biometric profiles and exercise templates using a structured knowledge base refined through reinforcement learning on population level usage data.

Actionable suggestions are then transmitted to the user via an interactive chatbot interface operating within the graphical user interface (GUI) of a mobile device or smart display. The chatbot retrieves programmatically generated recommendations from the progression engine and contextualizes them using natural language processing to produce intelligible prompts. For instance, the chatbot may display, “Your heart rate stayed flat during intervals. Ready to ramp up? Consider increasing incline by 2% or switching to sprints.” By combining real time biometric analysis, deviation detection algorithms, and decision logic driven prompts delivered through a non-generic chatbot interface, the system implements a concrete technological solution. This results in improved workout personalization, reduced manual input requirements, and enhanced responsiveness to intra session performance variability.

The AI Trainer chatbot described herein performs a technically structured transformation of data into contextualized user-facing adaptations through a machine-implemented process that extends beyond simple information presentation. The chatbot is part of a broader, multi-component system that includes data ingestion, processing, decision-making, and natural language generation subsystems, each of which contributes to improving the underlying computer functionality of adaptive fitness coaching platforms.

Upon collecting both subjective and objective metrics, such as user-entered ratings of perceived exertion (RPE) and biometric signals from wearable devices including heart rate, motion patterns, cadence, and variability metrics sampled between 1 Hz and 10 Hz, the system aggregates and interprets these inputs through a metric aggregation engine. These data are normalized using filtering techniques such as Kalman filters or low-pass smoothing filters, which ensure noise reduction and stable data patterns. For instance, overlapping heart rate zones may be defined for adaptive triggers, such as 120-140 bpm for moderate-intensity training or 140-160 bpm for anaerobic intervals. When the system detects that the user consistently remains in the lower half of an intended zone (e.g., 122-128 bpm in a 120-140 bpm target during interval sprints), a deviation flag is issued. The progression engine then evaluates this in light of other indicators like RPE falling below a threshold range of 3-5, motion vector consistency, and session completion history over a rolling 7-day window.

The decision logic produced by the progression engine is packaged into structured payloads which the chatbot receives. For example, if the user reports RPE values of 3.5 for squats for three consecutive sessions, and heart rate shows insufficient rise (e.g., plateauing at 118 bpm despite increased weights), the chatbot receives a data bundle with the fields exercise_type: squat, intensity_trend: flat, suggested_adjustment: +10% load or variation: goblet squat. The chatbot does not rely on hard-coded responses but uses a natural language generation (NLG) subsystem to dynamically generate prompts from these payloads. In this example, the chatbot might generate: “You're crushing your squats! Want to challenge yourself with a goblet squat or add 10% more weight to your next session?”

To further illustrate, the chatbot distinguishes between chronic and acute adaptations. In chronic scenarios, such as repeated plateauing HRV trends in the range of about 30 ms-45 ms over a 3-week period (where the target range is 50-70 ms), it may suggest a deload week. Alternatively, if acute deviations are detected, such as a single-session drop in heart rate recovery (e.g., delta HR recovery <12 bpm within 1 minute post-interval), the chatbot may issue a rest-based intervention prompt, such as: “Your recovery seems a bit slower today. Let's keep things moderate, reduce reps or take longer rests between sets.”

Importantly, the chatbot does not merely rephrase user input or output static suggestions. Instead, it operates as a dynamically adaptive, machine-enabled agent that translates multi-modal sensor input and user metadata (including pharmacological data, prior progress trends, and goal alignments) into contextualized guidance tailored to the user's training state. When a pharmacological tag is set (e.g., beta_blocker_user: true), the system reprioritizes subjective metrics such as RPE in the progression decision tree. In this case, the chatbot might generate: “Because your medication may limit heart rate changes, we're focusing more on how hard it feels. Ready to bump up difficulty if RPE has stayed low?”

These functionalities are grounded in technical implementation and improve the functioning of the overall system. By automating adaptation through structured rule execution, metric fusion, and logic-to-language transformation, the chatbot component provides a concrete application of machine-executed operations that materially alter the system's operation beyond mental steps or manual tracking.

### Multi-Source Data Fusion and Override Logic:

In some embodiments, the system is configured to perform multi-modal data fusion by integrating subjective self-reported input with objective sensor-based measurements in a rule-governed, adaptive decision engine. An example use case involves users whose biometric responses are pharmacologically attenuated, such as individuals prescribed beta blockers that suppress heart rate elevation. During the intake process, the system retrieves or infers pharmacological metadata tags, e.g., beta_blocker_user: true, which are stored as structured fields in the user's health profile. This metadata may be populated either through direct user responses to structured questionnaire elements (e.g., checkboxes or toggles confirming beta blocker usage), or via secure API-based integration with third-party electronic health records (EHR) or pharmacy data repositories.

Once the metadata tag is set, the system activates an override condition in the progression logic pipeline. Specifically, the logic associated with workload progression rules adjusts its weighting schema within the metric aggregation layer, elevating the priority of subjective metrics such as Rating of Perceived Exertion (RPE) over biometric signals such as heart rate. The metric aggregation layer employs a tunable weighting algorithm that maps each metric source to a confidence coefficient. For example, if a beta_blocker_user tag is active, the system may assign an 80-95% confidence weight to RPE data, and a 5-20% weight to heart rate trends, effectively rendering RPE the dominant driver of adaptation logic.

To further refine precision, the system applies normalization and smoothing algorithms (e.g., Exponential Moving Averages) to recent RPE entries associated with specific exercises, and compares these against the expected RPE values stored in the user's baseline model. When deviation thresholds are exceeded (e.g., RPE≤4 for three or more consecutive sessions of a specific movement), the rules-based progression engine invokes a lookup from the exercise library to retrieve candidate progressions. These progressions may include parameter modifications (e.g., increase load by 10-20%, increase volume by 1-2 sets), alternative exercise variants (e.g., switch from goblet squat to barbell front squat), or adjustments in rest intervals.

The AI Trainer chatbot then translates these backend-driven programmatic adaptations into natural language suggestions, contextualized by the user's pharmacological profile and exercise history. For example, a message may read: “We have noticed your perceived exertion has stayed low for squats, even with current intensity levels. Since your profile indicates heart rate suppression due to medication, we are using your feedback to drive adjustments. Would you like to increase weight by 15%, or try a more advanced squat variant?”

This approach improves the reliability and personalization of the training regimen in users with known biometric irregularities by incorporating dynamic metadata-driven overrides, structured multi-source weighting, and automated logic pipelines. The architecture addresses technical challenges of real-time adaptability, user-specific exception handling, and interface-level responsiveness. As such, it provides a concrete technological improvement beyond mere abstract mental processes or manual evaluation, and satisfies the machine-implemented requirements for patent eligibility.

### Prompting and Response Mechanism:

When an adaptation condition is met, based on a combination of subjective and objective performance metrics analyzed by the progression engine, the AI Trainer system dynamically generates a context-aware prompt using a structured, rule-based decision engine. This engine cross-references the user's real-time performance indicators, e.g., consistently low RPEs in the range of 2 to 4, or biometric data indicating sub-threshold heart rate response during targeted high effort zones, with a local suggestion database comprising pre-indexed messages and adaptation rules. These prompts are not static, but are constructed in real time using a prompt templating subsystem that dynamically selects variables such as the exercise name, e.g., “squat”, condition met, e.g., “low effort over 3 sessions”, and suggested alternatives, e.g., “add 10% weight”, “add 1 set”, or “switch to front squats”. Once the system confirms that an adaptation condition has been met, for example, the user has completed three sessions within a 7-day window with RPEs averaging below a predetermined progression threshold, e.g., RPE<4, the progression engine emits an event trigger to the chatbot interface. The chatbot then queries the decision map and constructs a prompt formatted as a natural language message using structured tags, such as: {exercise_type}, {performance_state}, {suggestion_1}, {suggestion_2}. This templated prompt is rendered through the GUI and presented to the user as an actionable selection menu.

Upon the user's response, e.g., selecting “Add 10% weight”, the AI Trainer backend stores this choice in the user's adaptive training profile, updating the exercise parameters and modifying the training plan for the next session. These selections are recorded in an interaction log, and the updated state is used as a new input feature vector for the next decision cycle. This feedback loop allows the prompt generation engine to maintain contextual continuity, reducing the need for user re-entry of preferences or feedback. For example, if the user repeatedly selects “increase load” while maintaining low RPEs, the system may begin to auto-recommend more aggressive progressions in future prompts, such as “Add 15% weight” or “Try deficit front squats”, based on evolving difficulty maps indexed by movement type and user history. This process illustrates a non-abstract implementation of computer functionality that transforms raw performance data into contextual, system-generated prompts via a series of machine-executed operations that go beyond mental steps or manual coaching. The structured logic and feedback integration represent an improvement in the functioning of a computer-based fitness adaptation system.

### System-Level Technical Benefits:

This system architecture delivers a technological improvement over conventional static fitness programs by implementing a non-generic, data-driven feedback loop that adapts in real-time to both physiological and perceptual signals. It reduces latency in training plan adjustment, improves precision by factoring individual biometric responses, and increases reliability by cross-referencing multiple data sources. The combined use of structured subjective inputs and high-resolution biometric feedback, fused and interpreted by a progression engine and surfaced through an intelligent chatbot interface, constitutes a concrete improvement to computer functionality and system responsiveness. Unlike manual plan adjustments or paper-based coaching, this implementation supports autonomous real-time personalization in a repeatable, scalable, and trackable manner.

### Multimodal Input and Systems Integration

In some embodiments, the system receives multimodal data inputs across several channels, each mapped to structured processing pipelines. Graphical user interface (GUI) input may include direct user interactions such as sliders for fatigue rating, dropdowns for workout preferences, and tap selections for pain or exertion zones, all timestamped and stored in a time-series data store. Wearable device input is collected via standard APIs (e.g., Bluetooth Low Energy protocols) from devices such as smartwatches or chest straps, and may include continuous real-time sampling of heart rate, heart rate variability, accelerometer and gyroscope data at sampling frequencies between 1 Hz and 10 Hz. This data is streamed to the system's metric aggregation layer, which performs normalization, smoothing (e.g., via Kalman filters), and segmentation of activity sessions.

Simultaneously, video data may be captured using a smartphone or webcam interface, with frames transmitted to the form detection engine. This engine utilizes spatial tracking algorithms in conjunction with convolutional neural networks (CNNs) trained on exercise-specific movement libraries. The CNNs detect skeletal landmarks (e.g., joint centers such as knees, hips, shoulders) and generate pose vectors over time. Deviations from biomechanical norms are encoded into a form deviation heatmap, which highlights inefficient movement patterns, e.g., anterior knee translation or trunk instability, and feeds into a programmatic rule engine for modification of the user's training plan. Textual input may also be collected via the AskAI chatbot, where users submit natural language descriptions of how they feel (e.g., “my back felt tight today” or “I had trouble balancing in lunges”). This text is parsed using an NLP pipeline that tags symptom entities, sentiment, and contextual cues. These cues are matched to pre-indexed conditions in a symptom-response database and linked to training adaptations (e.g., replace lunges with supported split squats, add hip mobility drills).

The system triangulates these inputs using a multimodal data fusion engine. For instance, if wearable data indicates lower-than-expected heart rate, and user RPE is also low, while video analysis shows form stability, the progression engine may recommend increasing workload. Conversely, if heart rate is suppressed but user is on beta blockers (as flagged by intake metadata), and RPE is high, the system may maintain or regress intensity. This real-time synthesis of subjective, biometric, and visual data reduces reliance on mental steps or human judgment and enables rapid adaptation of a tailored program, issued as a chatbot prompt rendered through the GUI (e.g., “Based on your low RPE and stable form, would you like to increase resistance?”). By tying sensor and user-generated data to actionable system logic, the claimed invention reflects a technical improvement in exercise programming systems. It implements a specific, non-generic integration of AI, signal processing, and real-time control over personalized training plans. This structured data processing workflow, mapped from device-layer input through algorithmic interpretation to GUI-rendered feedback.

### Explore Hub and Streamlined User Experience

To support user navigation and decision-making, the system includes an Explore Hub—an interface that aggregates workout templates, educational lessons, product recommendations, and available assessments. The Explore Hub is configured to surface resources based on user progress, history, and system-inferred need. For example, a user exhibiting decreased glute activation in assessment modules may be automatically directed to a resource pack containing glute-specific activation drills and anatomy education. The platform thus combines intelligent prompt-based automation with real-time decision trees, chatbot interactivity, biometric integration, and dynamic content delivery. This architecture provides an improvement in both the user experience and technical performance of exercise programming systems by reducing response latency, improving the precision of training adaptations, and increasing the reliability of prescribed outcomes across a diverse user base.

### Examples

Clause 1. A method executed by a health engine of a computing device for real-time fitness tracking and scheduling, the method comprising: providing the computing device comprising: an output device having a graphics processing unit in bidirectional communication with an interface bus; an interface controller in bidirectional communication with the interface bus; one or more data storage devices in communication with a health engine via the interface controller; and a peripheral interface having a serial interface controller in communication with a parallel interface controller, the parallel interface is in bidirectional communication with the interface bus; receiving, by the health engine, health data being at least a textual description of at least one of a location and an intensity of pain; receiving, from one or more wireless health devices, real-time biometric data of a user including at least one of a heart rate measurement, blood pressure measurement, or calories burned during a workout; displaying, via a graphical user interface (GUI) of the computing device, a graphic of the pain as pain points on a graphical representation of a human body based on an analysis by the health engine of the textual description of the pain; receiving, by the health engine, a first health goal of the user for completion during a first time period and a second health goal of the user for completion during a second time period, wherein the health data, the first health goal, and the second health goal are received from user input in response to a health questionnaire, wherein the first health goal is relieving pain, wherein the second health goal is to complete a sports event, wherein the second time period is a future time based on a sports event date, wherein the first time period is a duration involving a fitness schedule including a set of fitness activities to progress towards the first goal; executing, via a health engine, a heatmap component configured to provide a pictorial heatmap representation of muscle activation during an exercise comprising: pictorially representing, at the GUI of the computing device, a first set of muscles of the user engaging in an exercise that should be activated during the exercise using a first color, partially based on a user-specific physical characteristic from the health questionnaire; pictorially representing, at the GUI of the computing device, a second set of muscles of the user engaging in the exercise that should not be activated during the exercise using a second color, partially based on the user-specific physical characteristic from the health questionnaire; adapting the pictorial heatmap representation of muscle activation based on the user-specific physical characteristic, such that the pictorial heatmap representation is affected by the health questionnaire, wherein changing the exercise based on the location of the pain points if the pain points are located within an area of the second color of the pictorial representation; changing the exercise displayed on the GUI if the pain points are located within an area of the second color of the pictorial representation, based on the health engine determining that a current exercise is contraindicated; detecting, by the health engine, user activity data from the wireless health device in real time during exercise performance; dynamically updating, via the health engine, the pictorial heatmap representation as the health engine detects user engagement with the exercise based on the real-time user activity data to provide real-time data based on the adapted pictorial heatmap representation; receiving, by a form detection engine, a real-time video stream of a user performing a physical exercise, wherein the real-time video stream is captured by a client device; analyzing, by the form detection engine, a plurality of sequential video frames from the real-time video stream using a pose estimation algorithm to extract skeletal key points of the user; determining, based on the extracted skeletal key points, an exercise type being performed by the user and calculating joint angles and angular velocities over time; identifying, based on the calculated joint angles and angular velocities, one or more deviations between movement of a user and a reference biomechanical template corresponding to the exercise type; generating, by the form detection engine, a form deviation heatmap that visually depicts the one or more deviations on a human body outline, wherein a color-coded overlay is used to represent one or more of overactive, underactive, or misaligned muscle groups associated with the one or more deviations; and displaying, on a graphical user interface of the client device, the form deviation heatmap with corresponding muscle group indicators and real-time video or animation feedback; generating, by the health engine, a health profile comprising the health data, the first health goal, and the second health goal for the user; implementing, using an algorithm of the health engine, incorporating the health data in if-then scenarios to determine a first wellness action for the user to complete during the first time period in conjunction with the fitness schedule to achieve the first health goal, and wherein the first wellness action is a personalized education item that includes at least one of text, graphics, videos, and media, wherein the first wellness action based on the first health goal, and a second wellness action for the user to complete prior to the sports event date, and wherein the algorithm of the health engine is based on a hierarchy of skill development functions having a first level including one or more foundational elements, wherein the hierarchy has a logical order that builds upon the foundational elements and progresses to finer points at a second and third level of the hierarchy to form habits consistent with the first health goal and the second health goals; wherein the algorithm further evaluates the real-time biometric data to detect deviations from expected training intensity and adjusts the second wellness action based on the deviations; and creating, by the health engine, a health and wellness program in the health profile based on the first wellness action and the second wellness action.

Clause 2. The method of clause 1, wherein the health data is selected from the group consisting of: medical data, genetic data, nutritional data, fitness data, and environmental data.

Clause 3. The method of clause 2, wherein the medical data is selected from the group consisting of: a known health problem of the user, a prior health problem of the user, a current health problem of the user, a health problem of a family member associated with the user, and a physiological or biochemical measurement of the user.

Clause 4. The method of clause 3, wherein the physiological or biochemical measurement of the user is selected from the group consisting of: a heart rate measurement, a resting metabolic rate (RMR) measurement, an oxygen consumption (V02) level measurement, a weight measurement, a body fat measurement, a visceral fat measurement, a muscle mass measurement, a measurement of body water of the user, a body mass index (BMI) measurement, a bone mass measurement, and a blood glucose level measurement.

Clause 5. The method of clause 2, wherein the genetic data includes genomic information.

Clause 6. The method of clause 2, wherein the fitness data is selected from the group consisting of: a type of exercise routine engaged in by the user, a type of workout engaged in by the user, a length of time spent on the exercise routine, a length of time spent on the workout a number of calories burned during the exercise routine, a number of calories burned during the workout, a heart rate achieved during the exercise routine, and a heart rate achieved during the workout.

Clause 7. The method of clause 2, wherein the environmental data includes a lifestyle choice of the user, and wherein the lifestyle choice of the user is selected from the group consisting of: a sleep habit of the user, a type of learner the user is, a smoking habit of the user, and an alcohol intake habit of the user.

Clause 8. The method of clause 2, wherein the nutritional data includes information selected from the group consisting of: types of foods eaten by the user, a number of daily calories consumed by the user, a quantity of meals consumed daily by the user, a quantity of snacks consumed daily by the user, a type of snacks consumed daily by the user, a type of beverage consumed daily by the user, and a quantity of beverages consumed daily by the user.

Clause 9. The method of clause 1, wherein the first time period is a current time period, and wherein the second time period is a future time period.

Clause 10. The method of clause 1, wherein the health data pertaining to the user is received from one or more wireless health devices tracking one or more biometric parameters of the user.

Clause 11. The method of clause 1, further comprising dynamically updating the form deviation heatmap in real time as the user performs additional repetitions of the physical exercise, such that the displayed muscle group indicators reflect evolving deviations.

Clause 12. The method of clause 1, further comprising modifying the reference biomechanical template based on body-type metadata obtained from a pre-exercise intake questionnaire completed by the user.

Clause 13. The method of clause 1, further comprising detecting one or more specific deviation patterns from the exercise type and, in response, generating a corrective exercise recommendation selected from a predetermined list of mobility or activation drills.

Clause 14. The method of clause 13, wherein the corrective exercise recommendation is presented to the user in the form of an in-application message or as an adjustment to a workout schedule displayed by the graphical user interface, wherein the reference biomechanical template is selected based on one or more user-specific characteristics including limb length ratios, historical injury data, or fitness level, and wherein the pose estimation algorithm includes a convolutional neural network trained on labeled video datasets containing annotated joint trajectories.

Clause 15. The method of clause 1, further comprising calculating a confidence score for each of the one or more deviations, and suppressing visual indicators on the form deviation heatmap below a predefined threshold.

Clause 16. The method of clause 1, wherein the pose estimation algorithm comprises a spatial-temporal model that leverages optical flow between consecutive video frames to improve key point tracking fidelity, wherein the color-coded overlay within the form deviation heatmap includes a muscle activation region rendered in accordance with inferred electromyographic activity data derived from a joint motion pattern of a user.

Clause 17. The method of clause 1, further comprising receiving real-time biometric data from a wearable sensor worn by the user, and correlating the real-time biometric data with pose-derived metrics to refine accuracy of the form deviation heatmap, wherein the real-time biometric data includes heart rate, skin temperature, or an electromyography signal.

Clause 18. The method of clause 1, further comprising classifying the physical exercise as belonging to one of a plurality of predefined exercise families using a video classifier engine, wherein the video classifier engine includes temporal convolution layers and attention layers configured to improve classification accuracy for compound movement exercises.

Clause 19. The method of clause 1, wherein the graphical user interface enables the user to enter subjective feedback data regarding a rating of perceived exertion for the physical exercise, and wherein the subjective feedback data is used to adjust a level of difficulty of one or more subsequent exercises in a workout program, and wherein the color-coded overlay in the form deviation heatmap comprises a red gradient to indicate overactive muscle groups, a blue gradient to indicate underactive muscle groups, and a green region to indicate proper alignment.

Clause 20. The method of clause 1, further comprising: storing historical deviation data associated with the user in a database and training a personalization model to anticipate future form breakdowns based on indicators of fatigue or workout duration, and wherein the personalization model is configured to generate one or more real-time prompts instructing the user to pause or modify the physical exercise based on a predicted injury risk; and generating an assessment report that includes the form deviation heatmap, the one or more deviations, one or more corrective action recommendations, and links to one or more video tutorials.

### Computer System

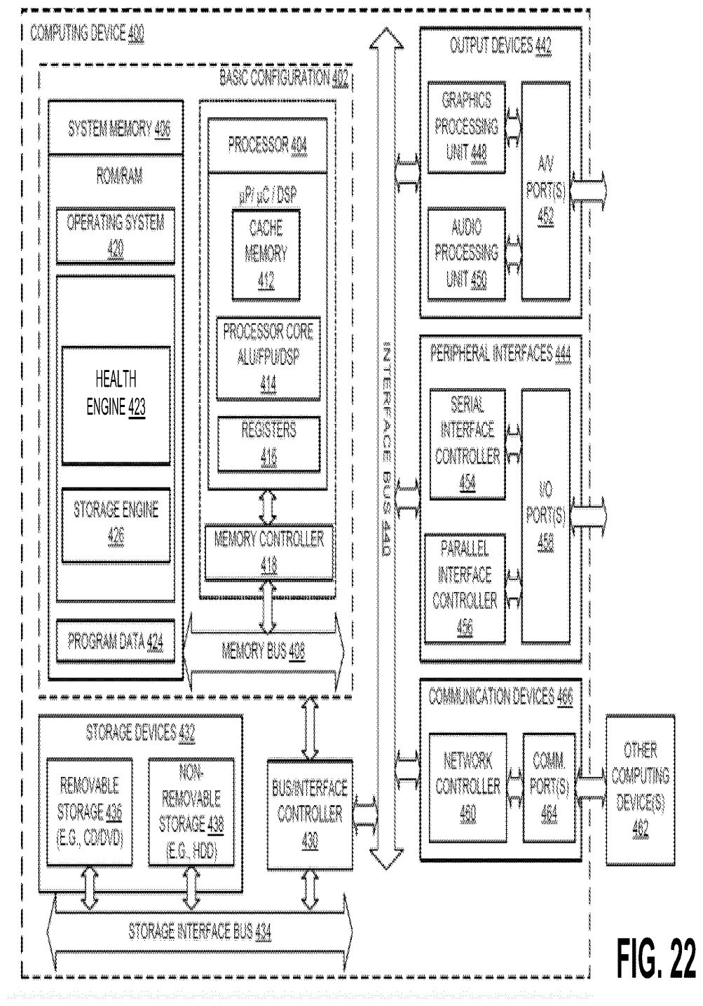

In some embodiments, the present invention may be a computer system, a method, and/or a computing device 104 (of FIG. 1) or 400 (of FIG. 22). For example, the computer system and/or the computing device 400 may be utilized to implement a method for real-time fitness tracking and scheduling.

A basic configuration 402 of a computing device 400 is illustrated in FIG. 22 by those components within the inner dashed line. In the basic configuration 402 of the computing device 400, the computing device 400 includes a processor 404 and a system memory 406. In some examples, the computing device 400 may include one or more processors and the system memory 406. A memory bus 408 is used for communicating between the one or more processors 10 404 and the system memory 406.

Depending on the desired configuration, the processor 404 may be of any type, including, but not limited to, a microprocessor (μP), a microcontroller (μC), and a digital signal processor (DSP), or any combination thereof. Further, the processor 404 may include one more levels of caching, such as a level cache memory 412, a processor core 414, and registers 416, among other 15 examples. The processor core 414 may include an arithmetic logic unit (ALU), a floating point unit (FPU), and/or a digital signal processing core (DSP Core), or any combination thereof. A memory controller 418 may be used with the processor 404, or, in some implementations, the memory controller 418 may be an internal part of the memory controller 404. 20

Depending on the desired configuration, the system memory 406 may be of any type, including, but not limited to, volatile memory (such as RAM), and/or non-volatile memory (such as ROM, flash memory, etc.), or any combination thereof. The system memory 406 includes an operating system 420, one or more engines, such as a health engine 423, and program data 424. In some embodiments, the health engine 423 may be a health application, a health software 29 5 10 15 20 program, a health service, or a health software platform. Moreover, in additional examples, the health engine 423 may comprise the algorithm 118.

The health engine 423 may receive the health data 108 pertaining to the user 102. The health data 108 may include medical data, genetic data, nutritional data, fitness data, and/or environmental data. The health engine 423 may also receive the first health goal 110 of the user 102 for completion during the first time period and the second health goal 112 of the user 102 for completion during the second time period. The health engine 423 may generate the health profile 116 for the user 102 that includes the health data 108, the first health goal 110, and the second health goal 112. Then, the health engine 423 may incorporate or implement, via the algorithm 118, the health data 108 in if-then scenarios 120 to determine the first wellness action 122 for the user 102 during the first time period to achieve the first health goal 110 and the second wellness action 124 for the user 102 during the second time period to achieve the second health goal 112. The health engine 423 may also create the health and wellness program 126 in the health profile 116 for the user 102 based on the first wellness action 122 and the second wellness action 124. Further, the computing device 400 may comprise a storage engine 426, which may be configured to store information, such as the health data 108, the first health goal 110 of the user 102, the second health goal 112 of the user 102, the health profile 116 for the user 102, the first wellness action 122 for the user 102, the second wellness action 124 for the user 102, and/or the health and wellness program 126, among other data not explicitly listed herein.

Moreover, the computing device 400 may have additional features or functionality, and additional interfaces to facilitate communications between the basic configuration 402 and any desired devices and interfaces. For example, a bus/interface controller 430 is used to facilitate communications between the basic configuration 402 and data storage devices 432 via a storage 30 5 10 15 20 interface bus 434. The data storage devices 432 may be one or more removable storage devices 436, one or more non-removable storage devices 438, or a combination thereof. Examples of the one or more removable storage devices 436 and the one or more non-removable storage devices 438 include magnetic disk devices (such as flexible disk drives and hard-disk drives (HDD)), optical disk drives (such as compact disk (CD) drives or digital versatile disk (DVD) drives), solid state drives (SSD), and tape drives, among others.

In some embodiments, an interface bus 440 facilitates communication from various interface devices (e.g., one or more output devices 442, one or more peripheral interfaces 444, and one or more communication devices 466) to the basic configuration 402 via the bus/interface controller 430. Some of the one or more output devices 442 include a graphics processing unit 448 and an audio processing unit 450, which are configured to communicate to various external devices, such as a display or speakers, via one or more A/V ports 452. The one or more peripheral interfaces 444 may include a serial interface controller 454 or a parallel interface controller 456, which are configured to communicate with external devices, such as input devices (e.g., a keyboard, a mouse, a pen, a voice input device, or a touch input device, etc.) or other peripheral devices (e.g., a printer or a scanner, etc.) via one or more I/O ports 458. Further, the one or more communication devices 466 may include a network controller 460, which is arranged to facilitate communication with one or more other computing devices 462 over a network communication link via one or more communication ports 464. The one or more other computing devices 462 include servers, the database 106, mobile devices, and comparable devices.

The network communication link is an example of a communication media. The communication media are typically embodied by the computer-readable instructions, data structures, program modules, or other data in a modulated data signal, such as a carrier wave or other transport mechanism, and include any information delivery media. A “modulated data signal” is a signal that has one or more of its characteristics set or changed in such a manner as to encode information in the signal. By way of example, and not limitation, the communication media may include wired media (such as a wired network or direct-wired connection) and wireless media (such as acoustic, radio frequency (RF), microwave, infrared (IR), and other wireless media). The term “computer-readable media,” as used herein, includes both storage media and communication media.

It should be appreciated that the system memory 406, the one or more removable storage devices 436, and the one or more non-removable storage devices 438 are examples of the computer-readable storage media. The computer-readable storage media is a tangible device that can retain and store instructions (e.g., program code) for use by an instruction execution device (e.g., the computing device 400). Any such, computer storage media is part of the computing device 400. The computer readable storage media/medium can be a tangible device that can retain and store instructions for use by an instruction execution device.

The computer readable storage media/medium may be, for example, but is not limited to, an electronic storage device, a magnetic storage device, an optical storage device, an electromagnetic storage device, and/or a semiconductor storage device, or any suitable combination of the foregoing. A non-exhaustive 20 list of more specific examples of the computer readable storage media/medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD32 5 10 15 ROM), a digital versatile disk (DVD), a memory stick, a floppy disk, and/or a mechanically encoded device (such as punch-cards or raised structures in a groove having instructions recorded thereon), and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Aspects of the present invention are described herein regarding illustrations and/or block diagrams of methods, computer systems, and computing devices according to embodiments of the invention. It will be understood that each block in the block diagrams, and combinations of the blocks, can be implemented by the computer-readable instructions (e.g., the program code).

The computer-readable instructions are provided to the processor 404 of a general purpose computer, special purpose computer, or other programmable data processing apparatus (e.g., the computing device 400) to produce a machine, such that the instructions, which execute via the processor 404 of the computer or other programmable data processing apparatus, create means for implementing the functions/acts specified in the block diagram blocks. These computer-readable instructions are also stored in a computer-readable storage medium that can direct a computer, a programmable data processing apparatus, and/or other devices to function in a particular manner, such that the computer-readable storage medium having instructions stored 20 therein comprises an article of manufacture including instructions, which implement aspects of the functions/acts specified in the block diagram blocks.

The computer-readable instructions (e.g., the program code) are also loaded onto a computer (e.g. the computing device 400), another programmable data processing apparatus, or 33 5 10 15 another device to cause a series of operational steps to be performed on the computer, the other programmable apparatus, or the other device to produce a computer implemented process, such that the instructions, which execute on the computer, the other programmable apparatus, or the other device, implement the functions/acts specified in the block diagram blocks

Computer readable program instructions described herein can also be downloaded to respective computing/processing devices from a computer readable storage medium or to an external computer or external storage device via a network (e.g., the Internet, a local area network, a wide area network, and/or a wireless network). The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming 20 languages, such as the “C” programming language or similar programming languages. The computer readable program instructions may execute entirely on the user's computer/computing device, partly on the user's computer/computing device, as a stand-alone software package, partly on the user's computer/computing device and partly on a remote computer/computing device or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to block diagrams of methods, computer systems, and computing devices according to embodiments of the invention. It will be understood that each block and combinations of blocks in the diagrams, can be implemented by the computer readable program instructions.

The block diagrams in the Figures illustrate the architecture, functionality, and operation of possible implementations of computer systems, methods, and computing devices according to various embodiments of the present invention. In this regard, each block in the block diagrams may represent a module, a segment, or a portion of executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block and combinations of blocks can be implemented by special purpose hardware based systems that perform the specified functions or acts or carry out combinations of special purpose hardware and computer instructions.

Another embodiment of the invention provides a method that performs the process steps on a subscription, advertising, and/or fee basis. That is, a service provider can offer to assist in the method steps for real-time fitness tracking and scheduling. In this case, the service provider can create, maintain, and/or support, etc. a computer infrastructure that performs the process steps for one or more customers. In return, the service provider can receive payment from the customer(s) under a subscription and/or fee agreement, and/or the service provider can receive payment from the sale of advertising content to one or more third parties.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others or ordinary skill in the art to understand the embodiments disclosed herein.

When introducing elements of the present disclosure or the embodiments thereof, the articles “a,” “an,” and “the” are intended to mean that there are one or more of the elements. Similarly, the adjective “another,” when used to introduce an element, is intended to mean one or 20 more elements. The terms “including” and “having” are intended to be inclusive such that there may be additional elements other than the listed elements.

Although this invention has been described with a certain degree of particularity, it is to be understood that the present disclosure has been made only by way of illustration and that 36 numerous changes in the details of construction and arrangement of parts may be resorted to without departing from the spirit and the scope of the invention.

What is claimed is:

1. A method executed by a health engine of a computing device for real-time fitness tracking and scheduling, the method comprising:
providing the computing device comprising:
an output device having a graphics processing unit in bidirectional communication with an interface bus;
an interface controller in bidirectional communication with the interface bus;
one or more data storage devices in communication with a health engine via the interface controller; and
a peripheral interface having a serial interface controller in communication with a parallel interface controller, the parallel interface is in bidirectional communication with the interface bus;
receiving, by the health engine, health data being at least a textual description of at least one of a location and an intensity of pain;
receiving, from one or more wireless health devices, real-time biometric data of a user including at least one of a heart rate measurement, blood pressure measurement, or calories burned during a workout;
displaying, via a graphical user interface (GUI) of the computing device, a graphic of the pain as pain points on a graphical representation of a human body based on an analysis by the health engine of the textual description of the pain;
receiving, by the health engine, a first health goal of the user for completion during a first time period and a second health goal of the user for completion during a second time period, wherein the health data, the first health goal, and the second health goal are received from user input in response to a health questionnaire, wherein the first health goal is relieving pain, wherein the second health goal is to complete a sports event,
wherein the second time period is a future time based on a sports event date, wherein the first time period is a duration involving a fitness schedule including a set of fitness activities to progress towards the first goal;
executing, via a health engine, a heatmap component configured to provide a pictorial heatmap representation of muscle activation during an exercise comprising:
pictorially representing, at the GUI of the computing device, a first set of muscles of the user engaging in an exercise that should be activated during the exercise using a first color, partially based on a user-specific physical characteristic from the health questionnaire;
pictorially representing, at the GUI of the computing device, a second set of muscles of the user engaging in the exercise that should not be activated during the exercise using a second color, partially based on the user-specific physical characteristic from the health questionnaire;
adapting the pictorial heatmap representation of muscle activation based on the user-specific physical characteristic, such that the pictorial heatmap representation is affected by the health questionnaire, wherein changing the exercise based on the location of the pain points if the pain points are located within an area of the second color of the pictorial representation;
changing the exercise displayed on the GUI if the pain points are located within an area of the second color of the pictorial representation, based on the health engine determining that a current exercise is contraindicated;
detecting, by the health engine, user activity data from the wireless health device in real time during exercise performance;
dynamically updating, via the health engine, the pictorial heatmap representation as the health engine detects user engagement with the exercise based on the real-time user activity data to provide real-time data based on the adapted pictorial heatmap representation;
receiving, by a form detection engine, a real-time video stream of a user performing a physical exercise, wherein the real-time video stream is captured by a client device;
analyzing, by the form detection engine, a plurality of sequential video frames from the real-time video stream using a pose estimation algorithm to extract skeletal key points of the user;
determining, based on the extracted skeletal key points, an exercise type being performed by the user and calculating joint angles and angular velocities over time;
identifying, based on the calculated joint angles and angular velocities, one or more deviations between movement of a user and a reference biomechanical template corresponding to the exercise type;
generating, by the form detection engine, a form deviation heatmap that visually depicts the one or more deviations on a human body outline,
wherein a color-coded overlay is used to represent one or more of overactive, underactive, or misaligned muscle groups associated with the one or more deviations; and
displaying, on a graphical user interface of the client device, the form deviation heatmap with corresponding muscle group indicators and real-time video or animation feedback;
generating, by the health engine, a health profile comprising the health data, the first health goal, and the second health goal for the user;
implementing, using an algorithm of the health engine, incorporating the health data in if-then scenarios to determine a first wellness action for the user to complete during the first time period in conjunction with the fitness schedule to achieve the first health goal, and
wherein the first wellness action is a personalized education item that includes at least one of text, graphics, videos, and media, wherein the first wellness action based on the first health goal, and a second wellness action for the user to complete prior to the sports event date, and
wherein the algorithm of the health engine is based on a hierarchy of skill development functions having a first level including one or more foundational elements,
wherein the hierarchy has a logical order that builds upon the foundational elements and progresses to finer points at a second and third level of the hierarchy to form habits consistent with the first health goal and the second health goals;
wherein the algorithm further evaluates the real-time biometric data to detect deviations from expected training intensity and adjusts the second wellness action based on the deviations; and
creating, by the health engine, a health and wellness program in the health profile based on the first wellness action and the second wellness action.

2. The method of claim 1, wherein the health data is selected from the group consisting of: medical data, genetic data, nutritional data, fitness data, and environmental data.

3. The method of claim 2, wherein the medical data is selected from the group consisting of:
a known health problem of the user, a prior health problem of the user, a current health problem of the user, a health problem of a family member associated with the user, and
a physiological or biochemical measurement of the user.

4. The method of claim 3, wherein the physiological or biochemical measurement of the user is selected from the group consisting of: a heart rate measurement, a resting metabolic rate (RMR) measurement, an oxygen consumption (V02) level measurement, a weight measurement, a body fat measurement, a visceral fat measurement, a muscle mass measurement, a measurement of body water of the user, a body mass index (BMI) measurement, a bone mass measurement, and a blood glucose level measurement.

5. The method of claim 2, wherein the genetic data includes genomic information.

6. The method of claim 2, wherein the fitness data is selected from the group consisting of: a type of exercise routine engaged in by the user, a type of workout engaged in by the user, a length of time spent on the exercise routine, a length of time spent on the workout a number of calories burned during the exercise routine, a number of calories burned during the workout, a heart rate achieved during the exercise routine, and a heart rate achieved during the workout.

7. The method of claim 2, wherein the environmental data includes a lifestyle choice of the user, and wherein the lifestyle choice of the user is selected from the group consisting of: a sleep habit of the user, a type of learner the user is, a smoking habit of the user, and an alcohol intake habit of the user.

8. The method of claim 2, wherein the nutritional data includes information selected from the group consisting of: types of foods eaten by the user, a number of daily calories consumed by the user, a quantity of meals consumed daily by the user, a quantity of snacks consumed daily by the user, a type of snacks consumed daily by the user, a type of beverage consumed daily by the user, and a quantity of beverages consumed daily by the user.

9. The method of claim 1, wherein the first time period is a current time period, and wherein the second time period is a future time period.

10. The method of claim 1, wherein the health data pertaining to the user is received from one or more wireless health devices tracking one or more biometric parameters of the user.

11. The method of claim 1, further comprising dynamically updating the form deviation heatmap in real time as the user performs additional repetitions of the physical exercise, such that the displayed muscle group indicators reflect evolving deviations.

12. The method of claim 1, further comprising modifying the reference biomechanical template based on body-type metadata obtained from a pre-exercise intake questionnaire completed by the user.

13. The method of claim 1, further comprising detecting one or more specific deviation patterns from the exercise type and, in response, generating a corrective exercise recommendation selected from a predetermined list of mobility or activation drills.

14. The method of claim 13, wherein the corrective exercise recommendation is presented to the user in the form of an in-application message or as an adjustment to a workout schedule displayed by the graphical user interface,
wherein the reference biomechanical template is selected based on one or more user-specific characteristics including limb length ratios, historical injury data, or fitness level, and
wherein the pose estimation algorithm includes a convolutional neural network trained on labeled video datasets containing annotated joint trajectories.

15. The method of claim 1, further comprising calculating a confidence score for each of the one or more deviations, and suppressing visual indicators on the form deviation heatmap below a predefined threshold.

16. The method of claim 1, wherein the pose estimation algorithm comprises a spatial-temporal model that leverages optical flow between consecutive video frames to improve key point tracking fidelity,
wherein the color-coded overlay within the form deviation heatmap includes a muscle activation region rendered in accordance with inferred electromyographic activity data derived from a joint motion pattern of a user.

17. The method of claim 1, further comprising receiving real-time biometric data from a wearable sensor worn by the user, and correlating the real-time biometric data with pose-derived metrics to refine accuracy of the form deviation heatmap,
wherein the real-time biometric data includes heart rate, skin temperature, or an electromyography signal.

18. The method of claim 1, further comprising classifying the physical exercise as belonging to one of a plurality of predefined exercise families using a video classifier engine,
wherein the video classifier engine includes temporal convolution layers and attention layers configured to improve classification accuracy for compound movement exercises.

19. The method of claim 1, wherein the graphical user interface enables the user to enter subjective feedback data regarding a rating of perceived exertion for the physical exercise, and wherein the subjective feedback data is used to adjust a level of difficulty of one or more subsequent exercises in a workout program, and
wherein the color-coded overlay in the form deviation heatmap comprises a red gradient to indicate overactive muscle groups, a blue gradient to indicate underactive muscle groups, and a green region to indicate proper alignment.

20. The method of claim 1, further comprising:
storing historical deviation data associated with the user in a database and training a personalization model to anticipate future form breakdowns based on indicators of fatigue or workout duration, and
wherein the personalization model is configured to generate one or more real-time prompts instructing the user to pause or modify the physical exercise based on a predicted injury risk; and
generating an assessment report that includes the form deviation heatmap, the one or more deviations, one or more corrective action recommendations, and links to one or more video tutorials.
